(19) United States
(12) Patent Application Publication
(10) Pub. No.: US 2023/0200989 A1
(43) Pub. Date: Jun. 29, 2023

(54) INTRODUCER SHEATH FIN DESIGN

(71) Applicant: EDWARDS LIFESCIENCES CORPORATION, Irvine, CA (US)

(72) Inventors: Brendan Christopher Cummings, Costa Mesa, CA (US); Ilan Tamir, Irvine, CA (US); Alyssa Meyer, Palo Alto, CA (US); Andrew Murphy, Campbell, CA (US); Matthew Davis, Auburn, CA (US); Stacy Patch, Santa Barbara, CA (US); Victor E. Viray, Sunnyvale, CA (US); John P. Lunsford, San Carlos, CA (US)

(21) Appl. No.: 18/115,583

(22) Filed: Feb. 28, 2023

Related U.S. Application Data
(63) Continuation of application No. PCT/US2021/048228, filed on Aug. 30, 2021.
(60) Provisional application No. 63/236,655, filed on Aug. 24, 2021.
(60) Provisional application No. 63/071,686, filed on Aug. 28, 2020.

(51) Int. Cl. A61F 2/24

(52) U.S. Cl. CPC A61F 2/2436

(57) ABSTRACT

Aspects of a sheath are disclosed herein including a radially expandable outer cylinder and including a plurality of sheath fins distributed circumferentially about the inner surface of the central lumen of the sheath. Each of the fins extends along a length of the inner surface of the sheath and when the sheath not expanded, the form a continuous inner surface of the sheath lumen. Aspects also include a sheath that includes a radially expandable cylindrical outer layer a stiff inner member, an inner liner layer coupled to the inner surface of the stiff inner member; and an outer liner layer disposed between an outer surface of the stiff inner member and an inner surface of the outer layer.

### CROSS-REFERENCE TO RELATED APPLICATIONS

This application is a continuation of International Application No. PCT/US2021/048228, filed Aug. 30, 2021, which claims the benefit of U.S. Provisional Application No. 63/071,686, filed Aug. 28, 2020, and U.S. Provisional Application No. 63/236,655, filed Aug. 24, 2021, the contents of which are incorporated herein by reference in their entirety.

### FIELD

The present application concerns examples of a sheath for use with catheter-based technologies for repairing and/or replacing heart valves, as well as for delivering an implant, such as a prosthetic valve to a heart via the patient's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques. Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient's vasculature (e.g., the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Introducer systems can be used in the delivery of prosthetic devices in the form of implantable heart valves, such as balloon-expandable implantable heart valves. An example of such an implantable heart valve is described in U.S. Pat. No. 5,411,552 entitled “Valve Prothesis for Implantation in the Body and a Catheter for Implanting such Valve Prosthesis,” and also in U.S. Pat. No. 9,393,110 entitled “Prosthetic Heart Valve,” both of which are hereby incorporated by reference. The introducer systems can also be used with the delivery systems for other types of implantable devices, such as self-expanding and mechanically-expanding implantable heart valves, stents or filters.

Conventional methods of accessing a vessel, such as a femoral artery, prior to introducing the delivery system include dilating the vessel using multiple dilators or sheaths that progressively increase in diameter. This repeated insertion and vessel dilation can increase the amount of time the procedure takes, as well as the risk of damage to the vessel.

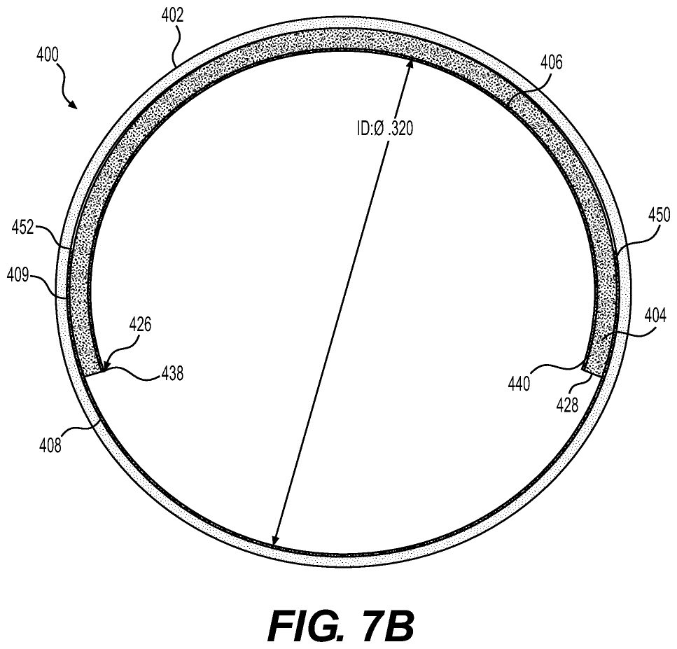

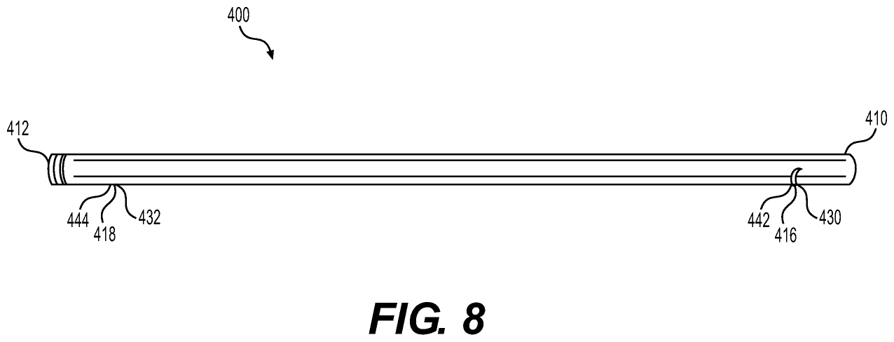

Expandable introducer sheaths, formed of highly elastomeric materials, allow for the dilating of the vessel to be performed by the passing prosthetic device. U.S. Pat. No. 8,790,387, which is entitled “Expandable Sheath for Introducing an Endovascular Delivery Device into a Body” and is incorporated herein by reference, discloses a sheath with a split outer polymeric tubular layer and an inner polymeric layer, for example in FIGS. 27A and 28. A portion of the inner polymeric layer extends through a gap created by the cut and can be compressed between the portions of the outer polymeric tubular layer. Upon expansion of the sheath, portions of the outer polymeric tubular layer have separated from one another, and the inner polymeric layer is expanded to a substantially cylindrical tube. Advantageously, the sheath disclosed in the '387 patent can temporarily expand for passage of implantable devices and then return to its starting diameter. This expansion is passive in nature, symmetric around the circumference of the sheath. The asymmetric expansion occurs dur to the unfolding of the inner polymeric layer into the gap formed in the outer polymeric tubular layer. This asymmetric expansion can result in unwanted stress on portions of the vessel adjacent the expanding portion and result in vessel trauma. Accordingly, there remains a need for further improvements in expandable introducer sheath for endovascular systems used to implant valves and other prosthetic devices.

### SUMMARY

The expandable sheath disclosed herein includes: a radially expandable cylindrical outer layer having a proximal end and a distal end, and defining a cylindrically shaped lumen extending longitudinally between the proximal end and the distal end, and having an inner surface; and a plurality of sheath fins distributed circumferentially about the inner surface and coupled thereto, wherein each of the sheath fins extends along a length of the inner surface of the outer layer, wherein the sheath is movable between an unexpanded state and an expanded state, and where in the unexpanded state the sheath fins form a continuous surface of the lumen of the outer layer.

Another example expandable sheath disclosed herein includes: a sheath comprising a radially expandable cylindrical outer layer having a proximal end and a distal end, and defining a cylindrically shaped lumen extending longitudinally between the proximal end and the distal end, and having an inner surface; and a plurality of sheath fins distributed circumferentially about the inner surface and coupled thereto, wherein each of the sheath fins extends along a length of the inner surface of the outer layer, wherein the sheath is movable between an unexpanded state and an expanded state, and wherein the unexpanded state the sheath fins form a continuous surface of the lumen of the outer layer; and an introducer sheath hub having a central lumen and a distal end, wherein the distal end of the introducer sheath hub is coupled to the proximal end of the introducer sheath, and where the central lumen of the sheath hub is coaxial with the central lumen of the introducer sheath.

A further expandable sheath disclosed herein includes: a radially expandable tube body having a proximal end a distal end, and defining a cylindrically shaped lumen extending longitudinally between the proximal end and the distal end, and having a wall thickness that extends between the proximal end and the distal end, and a plurality of wires distributed circumferentially within the side wall of the tube body, wherein each of the wires extend longitudinally between the proximal end and the distal end of the tube body, wherein the sheath is movable between an unexpanded state and an expanded state, and wherein the lumen has a uniform cylindrical shape in the unexpanded state, and the expanded state.

Another expandable sheath disclosed here in comprises: a sheath comprising: a radially expandable tube body having a proximal end a distal end, and defining a cylindrically shaped lumen extending longitudinally between the proximal end and the distal end, and having a wall thickness that extends between the proximal end and the distal end, and a plurality of wires distributed circumferentially within the sidewall of the tube body, wherein each of the wires extend longitudinally between the proximal end and the distal end of the tube body, wherein the sheath is movable between an unexpanded state and an expanded state, and wherein the inner lumen is a uniform cylindrical shape in the unexpanded and the expanded state; and an introducer sheath hub having a central lumen and a distal end, wherein the distal end of the introducer sheath hub is coupled to the proximal end of the introducer sheath, and wherein the central lumen of the sheath hub is coaxial with the central lumen of the introducer sheath.

The method of delivering a medical device (and/or a method of expanding an introducer sheath by a passing medical device) disclosed herein comprises: when delivering the medical device to a patient, inserting an introducer sheath into a blood vessel, the introducer sheath comprising: a radially expandable cylindrical outer layer having a proximal end and a distal end, and defining a cylindrically shaped lumen extending longitudinally between the proximal end and the distal end, and having an inner surface; and a plurality of sheath fins distributed circumferentially about the inner surface and coupled thereto, wherein each of the sheath fins extend along a length of the inner surface of the outer layer of the introducer sheath, where the sheath is movable between an unexpanded state and an and an expanded state, in the unexpanded state the sheath fins form an inner surface of the lumen of the outer layer; advancing a medical device through the lumen along an axis of the sheath and toward the distal end of the lumen; and expanding the lumen of the sheath while advancing the medical device through the introducer sheath, wherein the sheath expands symmetrically in the radial direction.

Another method of delivering a medical device (and/or a method of expanding an introducer sheath) disclosed herein comprises: when delivering the medical device to a patient, inserting an introducer sheath into a blood vessel, the introducer sheath comprising: a radially expandable cylindrical outer layer having a proximal end and a distal end, and defining a cylindrically shaped lumen extending longitudinally between the proximal end and the distal end, and having an inner surface; and a plurality of sheath fins distributed circumferentially about the inner surface and coupled thereto, wherein each of the sheath fins extend along a length of the inner surface of the outer layer of the introducer sheath, where the sheath is movable between an unexpanded state and an and an expanded state, in the unexpanded state the sheath fins form an inner surface of the lumen of the outer layer; advancing a medical device through the lumen along an axis of the sheath and toward the distal end of the lumen; and expanding the lumen of the sheath while advancing the medical device through the introducer sheath, wherein the sheath expands symmetrically in the radial direction.

A further method of delivering a medical device disclosed herein comprises: inserting an introducer sheath into a blood vessel, the introducer sheath comprising: a radially expandable tube body having a proximal end a distal end, and defining a cylindrically shaped lumen extending longitudinally between the proximal end and the distal end, and having a wall thickness that extends between the proximal end and the distal end, and a plurality of wires distributed circumferentially within the thickness of the tube body, wherein each of the wires extend longitudinally between the proximal end and the distal end of the tube body, wherein the sheath is movable between an unexpanded state and an expanded state, and wherein the inner lumen is a uniform cylinder in the unexpanded state, and the expanded state; advancing a medical device through the lumen along an axis of the sheath and toward the distal end of the lumen; and expanding the lumen of the sheath while advancing the medical device through the introducer sheath, wherein the sheath expands symmetrically in the radial direction.

Another example sheath disclosed herein comprises: a radially expandable cylindrical outer layer having a proximal end and a distal end, and defining a cylindrically shaped lumen extending longitudinally between the proximal end and the distal end, and having an inner surface; a stiff inner member having a proximal end and a distal end, an inner surface, and an outer surface opposite and spaced apart from the inner surface of the stiff inner member and each extending between the proximal end and the distal end of the stiff inner member, and the stiff inner member provided within at least a portion of the lumen of the outer layer; an inner liner layer coupled to the inner surface of the stiff inner member; and an outer liner layer disposed between the outer surface of the stiff inner member and the inner surface of the outer layer, wherein the sheath is movable between an unexpanded state and an expanded state, wherein in the unexpanded state, the inner liner layer forms a surface defining an inner lumen of the sheath, wherein in the expanded state, the inner liner layer and the outer liner layer each define a portion of the inner lumen of the sheath, and wherein the outer liner layer at least partially radially overlaps the stiff inner member in the unexpanded and expanded states.

An example sheath system disclosed herein comprises: a sheath including a radially expandable cylindrical outer layer having a proximal end and a distal end, and defining a cylindrically shaped lumen extending longitudinally between the proximal end and the distal end, and having an inner surface, a stiff inner member having a proximal end and a distal end, an inner surface, and an outer surface opposite and spaced apart from the inner surface of the stiff inner member and each extending between the proximal end and the distal end of the stiff inner member, and the stiff inner member provided within at least a portion of the lumen of the outer layer, an inner liner layer coupled to the inner surface of the stiff inner member, and an outer liner layer disposed between the outer surface of the stiff inner member and the inner surface of the outer layer; and an introducer sheath hub having a central lumen and a distal end, wherein the distal end of the introducer sheath hub is coupled to the proximal end of the sheath, and where the central lumen of the sheath hub is coaxial with the central lumen of the sheath, wherein the sheath is movable between an unexpanded state and an expanded state, wherein in the unexpanded state, the inner liner layer forms a surface defining an inner lumen of the sheath, wherein in the expanded state, the inner liner layer and the outer liner layer each define a portion of the inner lumen of the sheath, and wherein the outer liner layer at least partially radially overlaps the stiff inner member in the unexpanded and expanded states.

A further method of delivering a medical device (and/or a method of expanding an introducer sheath by a passing medical device) disclosed herein comprises: when delivering the medical device to a patient, inserting an introducer sheath into a blood vessel, the introducer sheath comprising: a radially expandable cylindrical outer layer having a proximal end and a distal end, and defining a cylindrically shaped lumen extending longitudinally between the proximal end and the distal end, and having an inner surface; a stiff inner member having a proximal end and a distal end, an inner surface, and an outer surface opposite and spaced apart from the inner surface of the stiff inner member and each extending between the proximal end and the distal end of the stiff inner member, and the stiff inner member provided within at least a portion of the lumen of the outer layer; an inner liner layer coupled to the inner surface of the stiff inner member, and an outer liner layer disposed between the outer surface of the stiff inner member and the inner surface of the outer layer, wherein the sheath is movable between an unexpanded state and an expanded state, wherein in the unexpanded state, the inner liner layer forms a surface defining an inner lumen of the sheath, wherein in the expanded state, the inner liner layer and the outer liner layer each define a portion of the inner lumen of the sheath, and wherein the outer liner layer at least partially radially overlaps the stiff inner member in the unexpanded and expanded states. The method further comprises advancing a medical device through the lumen along an axis of the sheath and toward the distal end of the lumen; and expanding the lumen of the sheath while advancing the medical device through the introducer sheath, wherein the sheath expands in the radial direction.

DESCRIPTION OF DRAWINGS

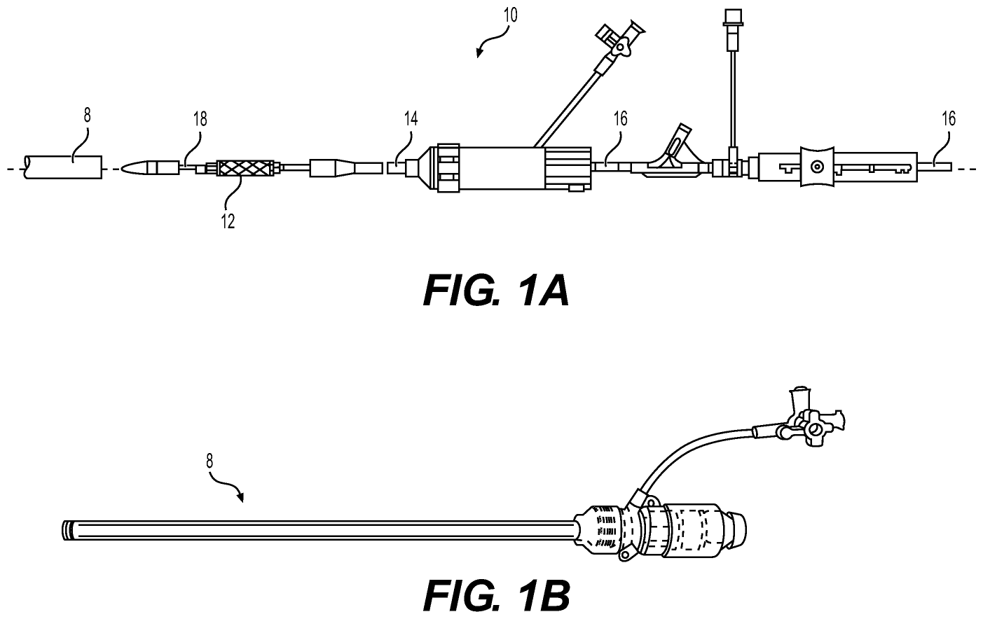

FIGS. 1A and 1B are side elevation views of an expandable introducer sheath (FIG. 1B) and a delivery apparatus for deployment through the sheath (FIG. 1A).

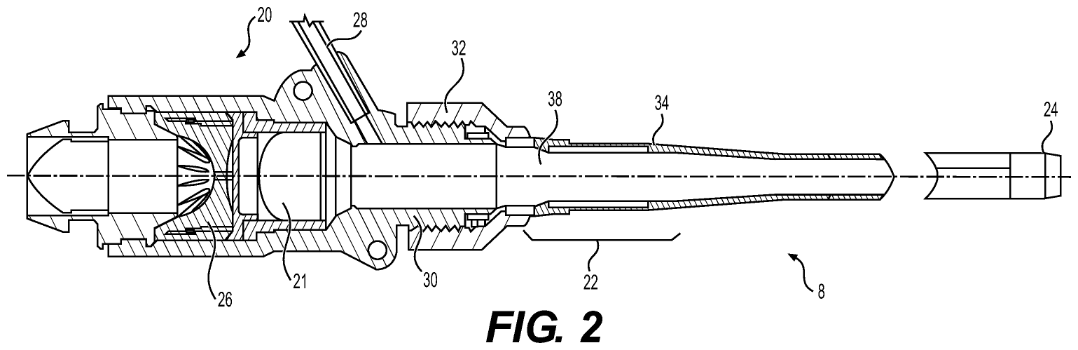

FIG. 2 is a side cross-sectional view of an example sheath and a hub.

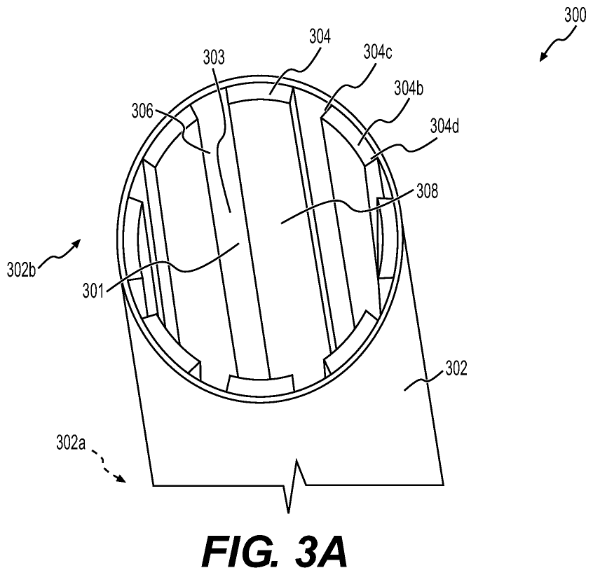

FIG. 3A is partial perspective view of the distal end of the sheath according to one implementation.

FIG. 3B is an enlarged view of a sheath fin of the sheath of FIG. 3A.

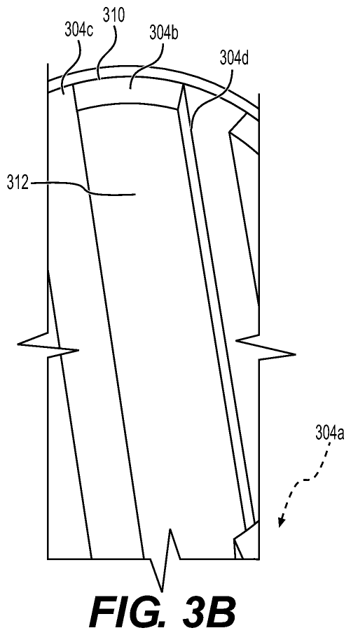

FIG. 4A is a cross-sectional view of the sheath of FIG. 3A in an unexpanded state.

FIG. 4B is a cross-sectional view of the sheath of FIG. 3A in the expanded state.

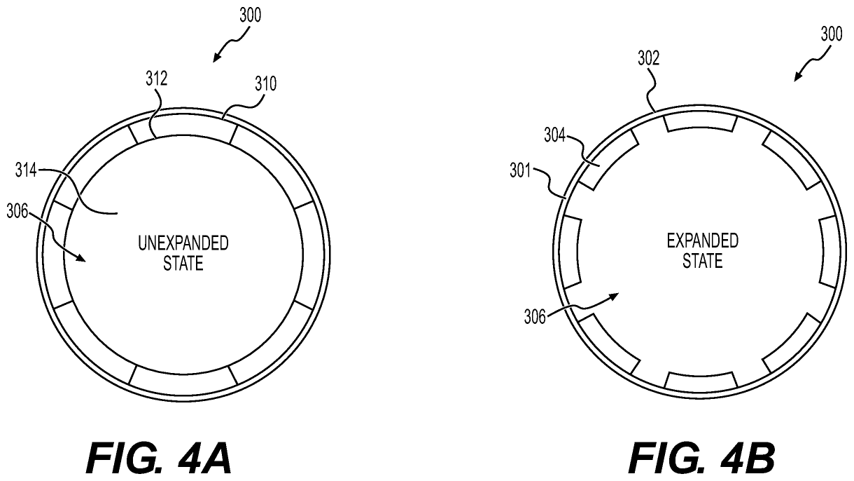

FIG. 5 is a partial perspective view of an example sheath according to another implementation.

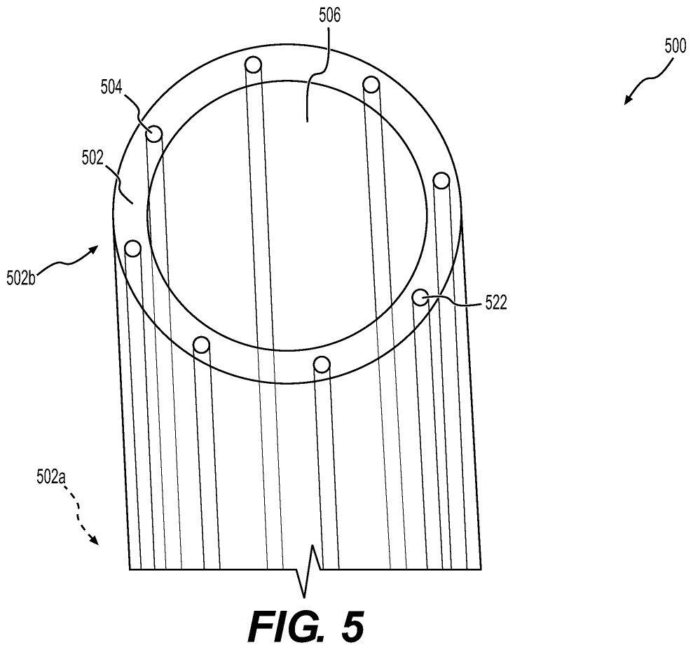

FIG. 6A is a cross-sectional view of the sheath of FIG. 5 in an unexpanded state.

FIG. 6B is a cross-sectional view of the sheath of FIG. 5 in an expanded state.

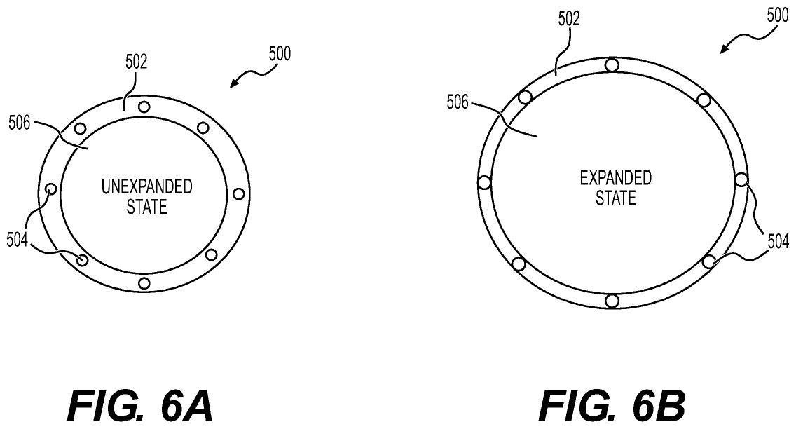

FIG. 7A shows a cross-sectional view of a sheath that has a stiff inner member, an inner liner layer and an outer liner layer in an unexpanded state.

FIG. 7B shows a cross-sectional view of the sheath of FIG. 7A that has a stiff inner member, an inner liner layer and an outer liner layer in an expanded state.

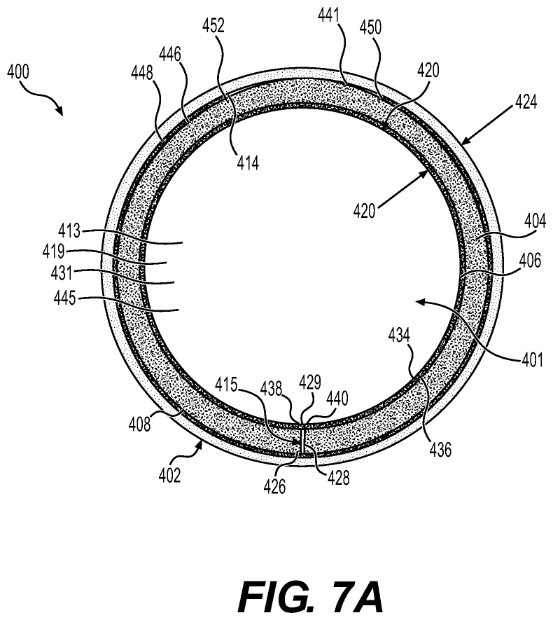

FIG. 8 shows a side view of the sheath of FIG. 7A.

### DETAILED DESCRIPTION

The following description of certain examples of the inventive concepts should not be used to limit the scope of the claims. Other examples, features, aspects, examples, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the examples of this disclosure are described herein. The described methods, systems, and apparatus should not be construed as limiting in any way. Instead, the present disclosure is directed toward all novel and nonobvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect or example of the present disclosure are to be understood to be applicable to any other aspect or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing examples. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

The disclosed expandable introducer sheath systems minimize trauma to the vessel by allowing for temporary, symmetric, expansion of a portion of the introducer sheath to accommodate the delivery system, followed by a return to the original diameter once the device passes through. During a transcatheter procedure, insertion and expansion of the introducer sheath causes the vessel walls to stretch radially, while insertion of the prosthetic device through the introducer causes the vessel walls to stretch longitudinally. When a passing prosthetic device stretches the sheath, the vessel walls are stretched in both directions simultaneously, which can lead to tearing. Disclosed examples of the introducer sheath systems allow for the symmetric expansion of the sheath. Tearing risk is minimized because radial pressure/stress is applied symmetrically resulting in corresponding symmetric expansion and stretching of the vessel wall. Some examples can comprise a sheath with a smaller profile than the profiles of prior art introducer sheaths. Furthermore, present examples can reduce the length of time a procedure takes, as well as reduce the risk of a longitudinal or radial vessel tear, or plaque dislodgement because only one sheath is required, rather than several different sizes of sheaths. Examples of the present expandable sheath can avoid the need for multiple insertions for the dilation of the vessel.

Disclosed herein are elongate introducer sheaths that are particularly suitable for delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, and also in U.S. Patent Application Publication No. 2012/0123529, both of which are hereby incorporated by reference. The expandable introducer sheaths disclosed herein may also be used to deliver other types of implantable medical device, such as self-expanding implantable heart valves, stents or filters. Beyond transcatheter heart valves, the active introducer sheath system 10 can be useful for other types of minimally invasive surgery, such as any surgery requiring introduction of an apparatus into a subject's vessel. For example, the active introducer sheath system 10 can be used to introduce other types of delivery apparatus for placing various types of intraluminal devices (e.g., stents, stented grafts, balloon catheters for angioplasty procedures, etc.) into many types of vascular and non-vascular body lumens (e.g., veins, arteries, esophagus, ducts of the biliary tree, intestine, urethra, fallopian tube, other endocrine or exocrine ducts, etc.). The term “implantable” as used herein is broadly defined to mean anything—prosthetic or not—that is delivered to a site within a body. A diagnostic device, for example, may be an implantable.

FIGS. 1A-1B illustrate an exemplary sheath 8 in use with a representative delivery apparatus 10, for delivering a prosthetic implant 12, such as a prosthetic heart valve or other type of implantable, to a patient. The delivery apparatus 10 described herein is exemplary only, and that other similar delivery systems can be used with the expandable sheath 8. The delivery apparatus 10 generally includes a steerable guide catheter 14 (also referred to as a flex catheter) and a balloon catheter 16 extending through the guide catheter 14.

The guide catheter 14 and the balloon catheter 16 illustrated in FIG. 1A are adapted to slide longitudinally relative to each other to facilitate delivery and positioning of the implant 12 at an implantation site in a patient's body, as described in detail below.

FIG. 1B illustrates an expandable introducer sheath 8 that is used to introduce the delivery apparatus 10 and the prosthetic device into the patient's body. Another example introducer sheath is described in U.S. patent Ser. No. 10/391,279, and also in U.S. Pat. No. 10,639,152, both of which are hereby incorporated by reference. As described herein, the sheath 8 has generally tubular configuration defining a central lumen to guide passage of the delivery system for the prosthetic heart valve. At a proximal end, the expandable introducer sheath 8 includes a hemostasis valve that prevents leakage of pressurized blood. Generally, during use a distal end of the sheath 8 is passed through the skin of the patient and inserted into a vessel, such as the femoral artery. The delivery apparatus 10 (with its implant) is then inserted into the sheath 8 through the hemostasis valve, and advanced through the patient's vasculature where the implant 12 is delivered and implanted within the patient. According to implementations described herein related to the sheath implementations 300, 400, 500 shown in FIGS. 3A-8 advancing a medical device along an axis of the sheath 300, 400, 500 expands a diameter of the sheath 300, 400, 500 from an unexpanded state to an expanded state. Portions of the sheath 300, 400, 500 may locally expand as a medical device (e.g., implant 12) is advanced from the proximal end to the distal end of the sheath 300, 400, 500.

In the implementation of FIGS. 3A-4B, advancing an implant/medical device through the sheath 300 effects a radially directed outward force against the elongated fins 304 extending along an inner surface of the sheath lumen. Expansion of the sheath 300 causes radial displacement and circumferential separation of the fins 304. In the implementation of FIG. 5, advancing an implant/medical device through the sheath 500 effects a radially directed outward force against the sheath 500 and causes radial displacement and circumferential separation of wires 502 embedded therein. In the implementation of FIG. 7A-7B, advancing an implant/medical device through the sheath 400 effects a radially directed outward force against the sheath 400, separating an inner member 404 and expanding the spacing between adjacent elongate edges of the inner member 404.

As shown in FIG. 2, the sheath 8 includes a hub 20, a flared proximal end 22 and a distal tip 24. The hub 20 is constructed of a rigid cylindrical structure defining a hub lumen 21 and houses a hemostasis valve 26 and may define a side port 28 and have a threaded distal end 30. The flared proximal end 22 of the sheath 8 includes a threaded female connector 32 mounted on a tubular wall structure 34. The tubular wall structure 34 is constructed from an elastic material and defines a central lumen 38 of the sheath 8 for receiving the delivery apparatus 10.

The hub 20 is attached to the flared proximal end 22 by twisting the threaded distal male end 30 into correspondingly threaded female connector 32. This places the hub lumen 21 in communication with the central lumen 38 of the tubular wall structure 34. The hemostasis valve 26 mediates access by the delivery apparatus 10 to the hub lumen 21 and central lumen 38 and ultimate deployment of the implant 12 in a pressurized (blood filled) environment. Side port 28 provides an additional access for application of saline or other fluids.

As shown in FIG. 2, the tubular wall structure of the sheath 8 has different layers. For example, a strain relief tubular layer can extend from the hub 20 towards the distal end of the sheath 8 along a length of the sheath. The strain relief tubular layer reduces material strain in the sheath 8 and reduces deformation when axial forces act on the sheath 8 during passage of the delivery apparatus 10 and implant 12. Generally, the strain relief tubular layer does not extend over the entire length of the sheath 8. The strain relief layer is preferable comprised of a relatively stiff material, such as HDPE, that can withstand the strains of the proximal end of the sheath 8 where it is joined to the hub 20 and other components for accepting initial insertion of the delivery apparatus 10. It terminates short of the distal end of the sheath 8 to facilitate a greater flexibility and lower profile of the distal end of the sheath 8.

The distal tip 24 provides some restraint to the otherwise radially expandable tubular wall structure 34. The distal tip 24 generally has a tubular structure with a slightly tapering or frusto-conical distal end. The distal tip 24 also helps with advancement over an introducer by providing a tapered advancement surface. Further the distal tip 24 improves the stiffness of the sheath 8 at its distal tip to guard against buckling or collapse of the tubular wall structure 34 during torque and advancement forces.

FIGS. 3A-8 illustrated various examples of the wall structure of the sheath 8. The introducer sheath 300, 400, 500 implementations of FIGS. 3A-8 described below include various structure/feature that ensure symmetric radial expansion of the sheath. As explained above, symmetric expansion minimizes trauma to the vessel because the outward radial pressure/forces resulting from the passing delivery apparatus 10 and/or implant 12 are applied evenly around the circumference of the vessel, allowing for uniform and even expansion (absent anatomical abnormality). The outer surface of each sheath 8, 300, 400, 500 seals onto the patient's blood vessel while the inner surface seals onto the delivery apparatus 10 or other device passing therethrough. As will be described in more detail below, symmetric expansion is facilitated by an elongated element coupled to or embedded in the elastic material of the sheath 300, 400, 500 wall structure. These elongated elements direct expansion of the sheath 300, 400, 500 at particular, equally spaced, locations around the circumference of the sheath 300, 400, 500, preventing uneven and/or asymmetrical expansion. The elastic material of the sheath body ensures that the sheath 300, 400, 500 is flexible and can conform to tortuous patient anatomy. However, the addition of the elongated elements retain adequate column strength so that the sheath 300, 400, 500 can be pushed through narrow vessels without significant force transmission loss.

FIGS. 3A-4B show a sheath 300 according to one implementation. The radially expandable sheath 300 defines a cylindrically shaped central lumen 306 extending therethrough and an opening at the distal end 300b allows passage of the implant 12 through the hub 20, the sheath, and to the treatment site. The proximal end 300a of the sheath 300 is coupled to the hub 20. The sheath 300 transitions between an unexpanded configuration (FIG. 4A) and an expanded configuration (FIG. 4B) to allow passage of an implant 12 and/or delivery apparatus 10 through the central lumen 306 of the sheath 300. In the unexpanded state, the outer diameter of the sheath 300 ranges between about 0.20 and about 0.30″. Ideally, in the unexpanded state the outer diameter of the sheath 300 is about 0.24″. In the expanded state, the outer diameter of the sheath 300 ranges between about 0.30″ to about 0.50″. Ideally, in the expanded state the outer diameter of the sheath 300 is about 0.40″.

The sheath 300 includes a tubular outer layer 302, a plurality of sheath fins 304 coupled to an inner surface 303 of the outer layer 302. As illustrated in FIG. 3A, the sheath fins 304 extend from the inner surface 303 towards the longitudinal axis of the sheath 300. As will be described in more detail below, the sheath 300 is movable between an unexpanded/non-expanded (FIG. 4A) and an expanded (FIG. 4B) configuration. In the unexpanded configuration, the inner surface 312 of the sheath fins 304 form a circumferentially continuous inner surface. During expansion, the spacing between the sheath fins 304 increases forming gaps 301 or spacing between adjacent sheath fins 304. The sheath fins 304 are coupled to the outer layer 302 and equally spaced around the circumference of the inner surface 303 of the outer layer 302, thereby allowing expansion of the sheath 300/outer layer 302 at those portions of the outer layer 302 between the sheath fins 304. As a result, the sheath 300 expands symmetrically in response to the radially directed outward force resulting from the passing delivery apparatus 10 and/or implant 12 against the inner surface 303 of the sheath 300 and/or inner surface of the sheath fins 304.

Each of the sheath fins 304 is coupled to the inner surface 303 of the outer layer 302. Each sheath fin 304 extends longitudinally and parallel to the longitudinal axis of the outer layer 302, forming (with the inner surface 312) the central lumen of the sheath 300. Each of the sheath fins 304 extend longitudinally along a length of the inner surface 312 of the outer layer 302. In some examples, the sheath fins 304 extend along a majority of the length of the inner surface 312. In other examples, the sheath fins 304 extend between the proximal end 300a and the distal end 300b of the sheath 300 and extend along the total length of the outer layer 302. It is contemplated that the length of various sheath fins 304 may be uniform or can vary around the circumference of the outer layer 302. As illustrated in FIGS. 3A-4B, the sheath 300 includes a plurality of sheath fins 304 equally spaced around the inner surface 302 of the outer layer 302. As provided in FIG. 3A, the sheath 300 includes eight sheath fins 304. However, it is contemplated that the sheath 300 can include additional or fewer sheath fins 304. As illustrated in FIG. 3A, the outer surfaces 310 of the sheath fins 304 are coupled to the inner surface 303 of the outer layer 302. The sheath fins 304 can be formed from silicone, a plastic or other suitable material. The sheath fins 304 can be integrally formed with the outer layer 302. For example, the sheath fins 304 can be coextruded with the outer layer 302. Alternatively, the sheath fins 304 can be coupled to the outer layer 302. For example, the sheath fins 304 can be coupled to the outer layer 302 by adhesive or other chemical fastener. In another example, the sheath fins 304 can be bonded to the outer layer 302 by a molding or heat treatment process.

The sheath fins 304 are arranged around the inner surface 303 such the sheath fins 304 abut each other forming a continuous surface of the lumen when the sheath is unexpanded. As illustrated in FIG. 4A, the sheath fins 304 form a continuous inner lumen 314, having a uniform radius about the circumference of the sheath 300. When the sheath is unexpanded, this inner lumen 314 extends longitudinally between the proximal end 302a of the sheath 300 to the distal end 302b of the sheath 300. As illustrated in FIG. 4B, the sheath 300 expands uniformly and symmetrically in the radial direction around the circumference of the sheath 300. As described above, the sheath fins 304 are coupled to the outer layer 302. As the sheath 300 expands, the portions of the outer layer 302 extending between adjacent fins 304 stretches and/or expands circumferentially while the portions of the outer layer 302 coupled to the sheath fins 304 does not stretch or expand. As a result, the sheath 300 expands symmetrically as outer layer 302 expands between the sheath fins 304 and the circumferential spacing between adjacent sheath fins 304 increases forming gaps 301. As illustrated in FIG. 4B, the width of the gap 301 in an expanded sheath is less than width of either of the adjacent sheath fins 304, where the width of the sheath fin 304 is measured between a leading edge 304c and a trailing edge 304d of the sheath fin 304. In some examples, when the sheath 300 is expanded, the width of the gap 301 formed between adjacent sheath fins 304 is at least half the width of one of the adjacent sheath fins 304. In another example, the width of the gap 301 is less than half the width of one of the adjacent sheath fins 304.

The sheath fins 304 of FIGS. 3A-4B each have a proximal end 304a adjacent a proximal end 302a of the outer layer 302 and a distal end 304b adjacent the distal end 302b of the outer layer 302, and a sheath fin body 308 which extends longitudinally between the proximal end 304a and the distal end 304b of each sheath fin 304. Each sheath fin body 308 has an outer surface 310 adjacent the inner surface 303 of the outer layer 302 and an inner surface 312 located between the outer surface 310 and the longitudinal axis of the sheath 300. In the example sheath 300 illustrated in FIGS. 3A-4B, sheath fins 304 each have an arcuate shaped in cross section. For example, both the inner and outer surfaces 312, 310 have a curved or arced shape in cross section. In some examples, the inner and outer surfaces 312, 310 have the same radius of curvature. In another example, the radius of curvature of the outer surface 310 of each of the sheath fins 304 is greater than the radius of curvature of the corresponding inner surface 314 of the sheath fins 304. As illustrated in FIGS. 4A and 4B, the outer surfaces 310 of each of the sheath fins 304 have the same radius of curvature. Likewise, the corresponding inner surfaces 312 of each of the sheath fins 304 have the same radius of curvature. Other cross sectional shaped fin 304 are contemplated. For example, the fins 304 can have a square, rectangular, hexagonal, trapezoidal, circular, elliptical, or any other regular or irregular shaped cross section. For example, the sheath fins 304 can have an arcuate-shaped outer surface 310 and a flat-shaped inner surface 314. The sheath fins 304 can have a uniform or varying cross sectional shape along a length of the fin. It is contemplated that all of the sheath fins 304 will have the same cross sectional shape. In another example, the cross sectional shape of at least one of the sheath fins 304 can vary from the remaining fins 304. Similarly, the material of the sheath fins 304 can be uniform or vary along a length of the fin 304.

As illustrated in FIG. 4A, when the sheath 300 is in the unexpanded state, each of the sheath fins 304 abuts an adjacent one of each of the sheath fins 304. Each sheath fin 304 has a longitudinally extending leading edge 304c and a longitudinally extending trailing edge 304d. In the aspect of FIG. 4A, the leading and trailing edges 304c, 304d each extend in a plane that intersects with the longitudinal axis of the sheath 300. In the unexpanded sate, the leading edge 304c of each of the sheath fins 304 abuts a trailing edge 304d of an adjacent one of the sheath fins 304. In general, the width of a sheath fin is measured between the leading and trailing edge 304c, 304d of the sheath fin 304. It is contemplated that the each of the sheath fins 304 will the same width. The width of the sheath fins 304 can remain constant or vary along a length of the sheath fin 304. Likewise, the width of the individual sheath fins 304 can vary around the circumference of the sheath 300.

As provided in FIGS. 3A-4B, the leading edge 304c and the trailing edge 304d of each of the sheath fins 304 form angled surfaces with respect to the outer surface 310 and the inner surface 312 of the sheath fin 304. The angled leading and trailing edges 304c, 304d allow each sheath fin 304 to abut an adjacent sheath fin 304 and forming a smooth (cylindrical) inner surface of the lumen 314 extending through the sheath 300.

When the sheath 300 transitions between the unexpanded and the expanded state, leading edge 304c of each of the sheath fins 304 slides along the trailing edge 304d of an adjacent fin 304. The leading and the trailing edges 304c, 304d can include a surface feature for promoting sliding movement between adjacent sheath fins 304 during expansion and contraction of the sheath 300. The surface feature can include a groove, a channel, a surface treatment, a lubricant, or a combination thereof.

Though not illustrated, it is contemplated that when the sheath 300 is in the unexpanded state each of the sheath fins 304 can be spaced apart from an adjacent sheath fin 304. In this example, in the unexpanded state, the initial width of the gap 310 between adjacent sheath fins 300 (e.g., a gap of less than half of a width of either of the adjacent sheath fins 304) would increase to a second, larger, gap width 301 upon expansion of the sheath 300.

The outer layer 302 has a cylindrical shape with a generally circular cross-section along its entire axial length. The outer layer 302 has an inner surface 303 which defines the central lumen 306 and extends longitudinally along the cylindrical cross-section of the outer layer 302. The outer layer 302 is sized to be received within the patient vasculature while also accommodating the size of the implant 12 to be delivered. Accordingly, it is desirable for the sheath 300 be easily expandable while having as reduced of profile as possible, to reduce trauma and prevent tearing of the patient's vasculature. As such, it is desirable to reduce the wall thickness of the outer layer 302 and the height/thickness of the sheath fins 304. The outer layer 302 can be formed from an elastomer. The outer layer 302 can also be formed from silicone, a plastic, or any other material suitable to form an elastic tubular layer. In some examples, the overall thickness between the inner surface of the sheath fin 304 and the outer surface of the outer layer 302 (i.e., the combined wall thickness of the outer layer 302 and the height/thickness of an adjacent sheath fin 304) ranges between about 0.04″ and about 0.07″. In some examples, the overall thickness is less than about 0.06″. In some examples, the outer layer 302 can have a wall thickness ranging between about 0.002″ and about 0.004″. In some examples, the outer layer can have a wall thickness of about 0.003″. It is contemplated that height/thickness the sheath fins 304, measured in a radial direction between the inner surface 312 of the sheath fin 304 and the inner surface 302 of the outer layer 302 can range between about 0.04″ and about 0.06″. In some examples, the sheath fins 304 have a height/thickness of about 0.05″.

The distal tip 24 (FIG. 2) of the sheath 300 provides an end cap sealing the fins within the sheath 300/outer layer 302. The distal tip 24/end cap is coupled to and/or integrally formed with the outer layer 302. The distal tip 24/end cap can be made of an elastomeric material. For example, the distal tip 24/end cap can be made of the same elastomeric material as the outer layer 302. The distal tip 24 can be formed from other plastics or any other material suitable to form a smooth catheter end surface for insertion into a patient's vascualtre. The distal tip 24 can form a c-shaped cross section and extends about the longitudinal axis of the sheath 300 to form a circular/ring shaped body at the end of the sheath 300. The distal tip 24/end cap can act to restrain the otherwise radially expandable sheath 300 while allowing the sheath 300 to expand during delivery and recapture of any device/implant delivered through the distal opening of the sheath.

As illustrated in FIGS. 3A-4B, the inner surface 312 of the sheath fins 304 defines an innermost surface of the sheath 300. As such, any radially directed outward force imposed from a passing delivery apparatus 10 and/or implant 12 is be applied to the inner surface 312 of the sheath fins 304. As described above, the outer 302 layer expands from an unexpanded state to an expanded state when a radial force acts on the sheath 300 from within the central lumen 306 (i.e., to the inner surface of the sheath fins 304). The sheath 300 retracts to the unexpanded state when the force is not acting on the sheath 300 from within the central lumen 306. It is contemplated that the outer layer 302 will locally expand at a local axial location to the expanded state at a corresponding location of the radial force within the central lumen 306. Likewise, the outer layer 302 will locally contract towards the unexpanded state when the radial force is not acting upon/removed from within the central lumen 306. This allows the majority of the sheath 300 to remain in the unexpanded state while inside the patient's vasculature while simultaneously accommodating a medical device being passed through the central lumen. This localized expansion allows for minimal expansion of a subject's vessels. When a circumferentially uniform instrument is inserted in the inner lumen 314 of the sheath 300, the sheath 300 expands to a uniform radius about the circumference of the sheath 300. This even/uniform expansion promotes smooth insertion and retraction from a subject and discourages internal laceration and uneven stretching of the blood vessels.

The sheath fins 304 also act to stiffen the outer layer 302 and provide column strength to the sheath 300. In an example sheath, the sheath fins 304 are stiffer than the elastic outer layer 304.

FIG. 5 shows a sheath 500 according to one implementation that includes a radially expandable tube body 502 having a plurality of wires 504 extending longitudinally within the sidewall of the tube body 502. As provided in FIG. 5, the plurality of wires 504 are arranged circumferentially around sidewall of the sheath and extend longitudinally between the proximal and distal ends 502a, 502b of the tube body 502. The sheath 500 transitions between an unexpanded configuration (FIG. 6A) and an expanded configuration (FIG. 6B) to allow passage of an implant 12 and/or delivery apparatus 10 through the central lumen of the sheath 500. In the unexpanded state, the outer diameter of the sheath 300 ranges between about 0.20 and about 0.30″. Ideally, in the unexpanded state the outer diameter of the sheath 300 is about 0.24″. In the expanded state, the outer diameter of the sheath 300 ranges between about 0.30″ to about 0.50″. Ideally, in the expanded state the outer diameter of the sheath 300 is about 0.40″.

The tube body 502 is formed from an elastomeric material that stretches easily to allow for expansion. The wires 504 are embedded in the tube body 502 and equally spaced around the circumference of the sheath 500, thereby directing symmetric expansion and discouraging deformation of the sheath 500/tube body 502 in response to the radially directed outward force provided by a passing delivery apparatus 10 and/or implant 12. The wires 504 also provide column strength such that the tube body 502 resists kinking during implantation at the treatment site. The embedded wires 504 are also visible under fluoroscopy allowing for ease in placement of the device. This fluoroscopic visibility of the wires 504 also allows the tube body 502 to be formed from a material having low radiopacity.

As shown in FIG. 5, the tube body 502 has a cylindrical shape with a circular cross-section along its entire length. The tube body 502 has an inner surface 501 which defines the central lumen 506 extending through the tube body 502, having a uniform cross-sectional shape in the expanded and unexpanded state.

As illustrated in FIGS. 6A-6B, the tube body 502 is configured to transition between an unexpanded state to expanded state when a radial force acts on the sheath 500 from within the central lumen 506, and sheath 500 retracts to the unexpanded state when the force is not acting on the sheath 500 from within the central lumen 506. As with sheath 300, it is contemplated that the tube body 502 will locally expand locally at a axial location corresponding to the location of the radial force applied from within the central lumen 506, and that the tube body 502 will locally contract towards the unexpanded state when the radial force is not acting upon/removed from within the central lumen 506. This allows the majority of the sheath 500 to remain in the unexpanded state, while inside the patient's vasculature and simultaneously accommodating a medical device being passed through the central lumen. This local expansion creates minimal expansion of a subject's vessels.

As described above and as shown in FIGS. 6A and 6B, the sheath 500 includes a plurality of the wires 504 embedded in the tube body 502. The wires 504 stiffen the tube body 502 but also allow for expansion of the sheath 500 by separating circumferentially upon expansion of the sheath 500. When an implant 12/delivery system 10 are inserted in the central lumen 506 of the sheath 500, the sheath 500 the position of the wires 504 around the circumference direct the uniform expansion of the sheath 500, preventing portions of the sheath 500 from protruding unevenly. This even expansion promotes smooth insertion and retraction from a subject and discourages internal laceration and uneven stretching of the blood vessels.

As illustrated in FIGS. 5-6B, the wires 504 are embedded within the sidewall of the tube body 502. Each of the wires 504 extend longitudinally and parallel to the longitudinal axis of the tube body 502. Each of the wires 504 extend longitudinally along a length of the tube body 502. In some examples, the wires 504 extend along a majority of the length of the tube body 502. In other examples, the wires 504 extend between the proximal and distal ends of the sheath 500 and extend along the total length of the tube body 502. It is contemplated that the length of various wires 504 may be uniform or can vary around the circumference of the tube body 502.

As illustrated in FIGS. 5-6B, the sheath 500 includes a plurality of wires 504 equally spaced around the tube body 502. As provided in FIG. 5, the sheath 500 includes eight sheath wires 504. However, it is contemplated that the sheath 500 can include additional or fewer wires 504. The wires 502 can be coupled or integrally formed with the tube body 502. For example, the wires 504 can be coextruded with the tube body 502. Alternatively, the wires 504 are fixedly coupled to the tube body 502. For example, the wires 504 can be coupled to the tube body 502 by adhesive or other chemical fastener. In another example, the wires 502 can be bonded to the tube body 502 by a molding or heat treatment process. In another example, the wires 502 are provided in corresponding wire holes 522. The diameter of the wire holes 522 may correspond to the diameter of the wires 504 or may be smaller than the diameter of the wires 504 such that the wires 504 are secured with in the wire holes 522 via friction fit.

Generally, the tube body 502 is formed from an elastomeric material (e.g., silicone) and the wires 504 are formed stiffer material. For example, the wires 502 can be formed from metal (e.g., stainless steel), a hard plastic, a composite, or other suitably stiff materials or combinations thereof. The wires 504 act to stiffen the tube body 502 and provide column strength to the sheath 500 while also ensuring even/symmetric radial expansion.

The wires 504 can have a uniform or varying circumference along their length. In an example sheath 500, the wires 504 have a diameter ranging between about 0.01″ and about 0.03″. In another example sheath 500, the wires 504 have a diameter ranging between about 0.020″ and about 0.025″. In a further example sheath 500, the diameter of the wires is about 0.020″. In another example sheath 500, the diameter of the wires is about 0.025″.

As illustrated in FIGS. 6A and 6B, the wires 504 have a circular shaped cross section, however other cross sectional shaped wires 504 are contemplated. For example, the wires 504 can have a square, rectangular, hexagonal, trapezoidal, torus, elliptical, or any other regular or irregular shaped cross section. It is further contemplated, that the sheath 500 can include various cross sectionally shaped wires 504.

As illustrated in FIGS. 6A and 6B, the wire 504 is fully embedded within the sidewall of the tube body 502. In another example (not shown), the wires 504 are partially embedded in the side wall of the tube body 502 and partially exposed to the inner lumen 506.

The tube body 502 has a cylindrical shape with a generally circular cross-section along its entire axial length and includes a central lumen 506 and extends longitudinally therethrough. The tube body 502 is sized to be received within the patient vasculature while also accommodating the size of the implant 12 to be delivered. Accordingly, it is desirable for the sheath 500 be easily expandable while having as reduced of profile as possible, to reduce trauma and prevent tearing of the patient's vasculature. As such, it is desirable to reduce the wall thickness of the tube body 502 and the diameter of the wires 504. In some examples, the wall thickness of the tube body 502 (including the wires 504) ranges between about 0.04″ and about 0.07″. In some examples, the overall thickness is less than 0.06″.

Similar to the sheath 300, the distal tip 24 (FIG. 2) of the sheath 500 provides an end cap sealing the wires 504 within the sheath 500/tube body 502. The distal tip 24/end cap is coupled to and/or integrally formed with the tube body 502. The distal tip 24/end cap can be made of an elastomeric material. For example, the distal tip 24/end cap can be made of the same elastomeric material as the tube body 502. The distal tip 24 can be formed from other plastics or any other material suitable to form a smooth catheter end surface for insertion into a subject The distal tip 24 can form a c-shaped cross section and extends about the longitudinal axis of the sheath 500 to form a circular/ring shaped body at the end of the sheath 500. The distal tip 24/end cap can act to restrain the otherwise radially expandable sheath 500 while allowing the sheath 500 to expand during delivery and recapture of any device/implant delivered through the distal opening of the sheath.

As described above, the expandable sheath 300, 400, 500 can be used to deliver, remove, repair, and/or replace a prosthetic device. In one example, the sheath described above can be used to deliver a prosthetic heart valve to a patient. For example, after the sheath is inserted into the body and into the patent's vasculature, a heart valve (in a crimped or compressed state) mounted on the distal end portion of an elongated delivery catheter is inserted into the sheath. Next, the delivery catheter and heart valve can be advanced through the sheath and through the patient's vasculature to the treatment site, where the valve is implanted.

When using the sheath 300 depicted in FIGS. 3A-4B, as the implant is passed through the outer layer 302, the implant exerts a radially directed outward force against the inner surface 312 of the sheath fins 304. This force causes the radial displacement and circumferential separation of the sheath fins 304 and drives expansion of the sheath 300. As the sheath 300 expands, the portions of the outer layer 302 extending between adjacent fins 304 stretches and/or expands circumferentially while the portions of the outer layer 302 coupled to the sheath fins 304 does not stretch or expand. As a result, the sheath 300 expands symmetrically as outer layer 302 expands between the sheath fins 304 forming gaps 301. The distal tip 28 and sheath 300 can expand again during retrieval of the delivery device or retrieved implant to easily receive the deflated balloon or retrieved implant.

When using the sheath 500 depicted in FIGS. 5-6B, as the implant is passed through the tube body 502, the implant exerts a radially directed outward force against the inner surface 501 of the sheath 500/tube body 502. This force causes the radial displacement and circumferential separation of the wires 504 and drives expansion of the sheath 500. As the sheath 500 expands, the portions of the tube body 502 extending between adjacent wires 504 stretches and/or expands uniformly around the circumference of the sheath 500. As a result, the sheath 500 expands symmetrically preventing unwanted the stress and trauma associated with asymmetric expansion. The distal tip 28 and sheath 500 can expand again during retrieval of the delivery device or retrieved implant to easily receive the deflated balloon or retrieved implant.

Further disclosed herein are examples of a sheath including a cylindrical outer layer, a stiff inner layer, an inner liner layer, and an outer liner layer. As described in more detail below, the stiff inner layer provides a stiff body for the sheath. The inner liner layer and the outer liner layer conform to the outer layer and/or stiff inner layer so that the inner liner layer provides a lubricious inner surface to define a channel for a medical device when the sheath is in an unexpanded state. The inner liner layer in combination with the outer liner layer provide a lubricious inner surface to define the channel for a medical device when the sheath is in an expanded state. The sheath is couplable to the sheath hub 20 and can be integrated into the sheath system as the exemplary sheath shown in FIGS. 1A-2 and described above.

FIGS. 7A-8 illustrate a sheath 400 that includes a cylindrical outer layer 402, a stiff inner layer 404, an inner liner layer 406, and an outer liner layer 408. The radially expandable cylindrical outer layer 402 has an inner surface 414 and defines a cylindrically shaped lumen 401. The stiff inner member 404 has an inner surface 420 and an outer surface 424. As will be described in more detail below, the stiff inner member 404 is disposed within at least a portion of the lumen 413 of the cylindrical outer layer 402. The inner liner layer 406 is coupled to the inner surface 420 of the stiff inner member 404. The outer liner layer 408 is disposed between the outer surface 424 of the stiff inner member 404 and the inner surface 446 of the outer layer 402.

The sheath 400 is movable between an unexpanded state and an expanded state. In the unexpanded state, as shown in FIG. 7A, the inner liner layer 406 forms a surface defining an inner lumen 401 of the sheath 400. In the expanded state, as shown in FIG. 7B, the inner liner layer 406 and the outer liner layer 408 each define a portion of the inner lumen 401 of the sheath 400. A portion of the outer liner layer 408 at least partially radially overlaps the outer surface of the stiff inner member 404 in the unexpanded and expanded states (e.g., portion 409 provided between outer layer 402 and inner layer 404 when the sheath is in the expanded state). As such, a portion of the outer liner layer 408 is always disposed between the outer layer 402 and the stiff inner member 404.

The radially expandable cylindrical outer layer 402 provides an outer body that is elastically expandable and interfaces with a patient as the sheath/medical device passes therethrough. The outer layer 402 has a proximal end 410 and a distal end 412 and defines a cylindrically shaped lumen 413 extending therethrough. The lumen 413 extends longitudinally between the proximal end 410 and the distal end 412 and has an inner surface 414. In some examples, the radially expandable outer layer 402 is formed as the outer layer 302 and alternative examples described above. In the unexpanded sate, the outer layer 402 has an outer diameter ranging from about 0.188 inches to about 0.288 inches. In some examples, the outer diameter of the outer layer 402 in the unexpanded state ranges from about 0.236 inches to about 0.240 inches.

The stiff inner member 404 provides radial stiffness for the sheath with respect to a central axis of the stiff inner member 404. As described above, the stiff inner member 404 is a cylindrical member that includes a slit 415 that extends longitudinally therethrough. In an unexpanded state, the stiff inner member 404 defines a cylindrically-shaped lumen 419. In an expanded state, the stiff inner member 404 forms a semicircular shape that defines a portion of the inner lumen 401 of the sheath 400. The stiff inner member 404 has a proximal end 416 and a distal end 418, an inner surface 420, and an outer surface 424 opposite and spaced apart from the inner surface 420 of the stiff inner member 404. The inner surface 420 and the outer surface 424 each extend between the proximal end 416 and the distal end 418 of the stiff inner member 404. The slit 415 extends between the proximal end 416 and the distal end 418 of the stiff inner member 404 and defines a first edge 426 and a second edge 428 of the stiff inner member 404. The first edge 426 of the stiff inner member 404 is adjacent the second edge 428 of the stiff inner member 404 when the sheath is in the unexpanded state. In some examples, the first edge 426 and the second edge 428 abut each other when the sheath 400 is in the unexpanded state. In the example shown in FIG.7A, the first edge 426 and the second edge 428 of the stiff inner member 404 are separated by a minimal circumferential distance. In the example shown in FIG. 7B, as the stiff inner member layer 404 expands to accommodate a medical device passing through the inner lumen 401, the width of the slit 415 increases and the first edge 426 and the second edge 428 of the stiff inner member 404 are separated by a circumferential distance. As such, in the example illustrated in FIGS. 7A-8, the circumferential distance between the first edge 246 and the second edge 248 in the expanded state is greater than a circumferential distance between the first edge 426 and the second edge 428 when the sheath 400 is in the unexpanded state. The stiff inner member 404 extends about a smaller fraction/portion of the circumference of the inner lumen 401 when in the expanded state than when in the unexpanded state. During expansion, the arc length between the first and second edges 426, 428 of the stiff inner member 404 stays about constant while radius of a semicircle formed by the cross section of the stiff inner member 404 increases.

The stiff inner member 404 has an inner diameter ranging from about 0.150 inches to about 0.226 inches in the unexpanded state. In some examples, the stiff inner member 404 has an inner diameter of about 0.185 inches when the sheath is in the unexpanded state. The stiff inner member 404 has a thickness ranging from about 0.0112 inches to about 0.0168 inches extending between the inner surface 420 and the outer surface 424. In some examples, the stiff inner member 404 has a thickness of about 0.014 inches. Accordingly, in some examples, the inner liner layer 406 has a diameter of about 0.118 inches when the sheath is in the unexpanded state. The stiff inner member 404 is formed from a thermoplastic polymer. In some examples, the stiff inner member 404 is formed from High Density Polyethylene (HDPE). The stiff inner member 404 is provided within at least a portion of the lumen 413 of the outer layer 402. The stiff inner member 404 extends between a longitudinal position spaced from the proximal and distal ends 410, 412 of the outer layer 402. In some examples, the stiff inner member 404 is spaced from about 0.4 inches to about 0.6 inches proximal of the distal end 412 of the outer layer 402 and from about 0.4 inches to about 0.6 inches distal of the proximal end 410 of the outer layer 402. For example, the stiff inner member 404 is spaced about 0.5 inches proximal of the distal end 412 of the outer layer 402 and about 0.5 inches distal of the proximal end 410 of the outer layer 402.

Although in the examples shown in FIGS. 7A-8, the stiff inner member 404 has an inner diameter of about 0.185 inches, in other examples the stiff inner member 404 has any diameter from 0.150 inches to 0.226 inches or any other diameter suitable to provide radial stiffness to a catheter and provide a lumen for a prosthetic to pass through. Although in the examples shown in FIGS. 7A-8 the stiff inner member 404 has a thickness of 0.014 inches, in other examples the stiff inner member has any thickness from 0.0112 inches to 0.0168 inches. Although in the examples shown in FIGS. 7A-8, the stiff inner member 404 is formed from HDPE, in other examples the stiff inner member 404 is formed from polypropylene or any other material suitable to provide radial stiffness for an expandable sheath. Although in the examples shown in FIGS. 7A-8, the stiff inner member 404 is spaced 0.5 inches proximal of the distal end 412 of the outer layer 402 and 0.5 inches distal of the proximal end 410 of the outer layer 402, in some examples, the stiff inner member 404 is spaced any length up to 0.6 inches proximal of the distal end 412 of the outer layer 402 and up to 0.6 inches distal of the proximal end 410 of the outer layer 402. In some examples the stiff inner member 404 extends the entire length of the outer layer 402.

The inner liner layer 406 provides a lubricious surface for a medical device such as a tool or a prosthetic to pass over when passing through the sheath 400. The inner liner layer 406 is a cylindrical layer that includes a slit 429 that extends longitudinally through the inner liner layer 406 such that in an unexpanded state the liner defines a cylindrical lumen 431 and is expandable to form a semicircular shape that defines a portion of the inner lumen 401 of the sheath 400. The inner liner layer 406 has a coefficient of friction that promotes smooth passage of a medical device through the inner lumen 401 and is less than the coefficient of friction of the stiff inner member 404. The inner liner layer 406 has a proximal end 430 and a distal end 432, an inner surface 434, and an outer surface 436 opposite and spaced apart from the inner surface 434 of the inner liner layer 406. The inner surface 434 and the outer surface 436 each extend between the proximal end 430 and the distal end 432 of the inner liner layer 406. The slit 429 that extends between the proximal end 430 and the distal end 432 of the inner liner layer 406. The slit 429 defined by a first edge 438 and a second edge 440 of the inner liner layer 406. The first edge 438 of the inner layer liner 406 is located adjacent the first edge 426 of the stiff inner member 408, and the second edge 440 of the inner liner layer 406 is located adjacent the second edge 428 of the stiff inner member 404. The first edge 438 of the inner liner layer 406 is adjacent the second edge 440 of the inner liner layer 406 when the sheath is in the unexpanded state. In some examples, the first edge 438 and the second edge 440 abut each other when the sheath is in the unexpanded state. In the example shown in FIG.7A, and as described above with respect to the stiff inner member 404, the first edge 438 and the second edge 440 of the inner liner layer 406 are separated by a minimal circumferential distance/gap when the sheath is in the unexpanded state. In the example shown in FIG. 7B, as the stiff inner member 404 and the inner liner layer 406 expand to accommodate a medical device passing through the inner lumen 401, the width of the slit 429 increases and the first edge 438 and the second edge 440 of the inner liner layer 406 are separated by a circumferential distance. As such, in the example illustrated in FIGS. 7A-8, the circumferential distance in the expanded state is greater than a circumferential distance between the first edge 438 and second edge 440 when the sheath is in the unexpanded state. The inner liner layer 406 extends about a smaller fraction/portion of the circumference of the inner lumen 401 when in the expanded state than when in the unexpanded state. During expansion, the arc length between the first edge 438 and the second edge 440 of the inner liner layer 406 stays about constant while radius of a semicircle formed by the cross section of the inner liner layer 406 increases.

The inner liner layer 406 has a diameter of 0.188 inches when the sheath is in the unexpanded state. The inner liner layer 406 has a radial thickness of 0.0015 inches. The inner liner layer 406 is disposed within the portion of the cylindrical lumen 419 defined by the stiff inner member 404, and the outer surface 436 of the inner liner layer 406 abuts and is coupled to the inner surface 420 of the stiff inner member 404 such that the inner liner layer 406 expands and contracts simultaneously with the stiff inner member 404.

Although in the examples shown in FIGS. 7A-8, the inner liner layer 406 has an inner diameter ranging from about 0.150 inches to about 0.226 inches in the unexpanded state. In some examples, the inner liner layer 406 has an inner diameter of about 0.188 inches. In other examples, the inner liner has any thickness from 0.001 to 0.005 inches or any other thickness suitable to resist folding upon movement between the unexpanded state and the expanded state. For example, the inner liner layer 406 has a thickness of about 0.0015 inches. Although in the examples shown in FIGS. 7A-8, the inner liner layer 406 is spaced proximal (e.g., 0.5 inches) of the distal end 432 of the outer layer 402 and spaced distal (e.g., 0.5 inches) of the proximal end 410 of the outer layer 402, in some examples, the inner liner layer 406 is spaced any length up to 0.6 inches proximal of the distal end 412 of the outer layer 402 and up to 0.6 inches distal of the proximal end 410 of the outer layer 402. In some examples, the inner liner layer 406 extends the entire length of the outer layer 402. Although in the example shown in FIGS. 7A-8 the inner liner layer 406 is coupled to the stiff inner member 404, in other examples, the inner liner layer 406 is coupled directly to the outer layer 402, or any other portion of the sheath suitable to anchor the inner liner layer 406.

The outer liner layer 408 provides a lubricious surface for a medical device such as a tool or a prosthetic to pass over when the sheath is in the expanded state. The outer liner layer 408 is a cylindrical layer that includes a slit 441 that extends longitudinally therethrough such that the outer liner layer 408 in an unexpanded state defines a semi cylindrical lumen 445 and expands to form a crescent shape that defines a portion of the inner lumen 401 of the sheath 400. The outer liner layer 408 has a coefficient of friction such that it promotes smooth passage of a medical device through the inner lumen 401 of the sheath 400 and is less than the coefficient of friction of the stiff inner member 404. The outer liner layer 408 has a proximal end 442 and a distal end 444, an inner surface 446, and an outer surface 448 opposite and spaced apart from the inner surface 446 of the outer liner layer 408. The inner surface 446 and the outer surface 448 each extend between the proximal end 442 and the distal end 444 of the outer liner layer 408. The slit 441 that extends between the proximal end 442 and the distal end 444 of the outer liner layer 408 that defines a first edge 450 and a second edge 452 of the outer liner layer 408. In the unexpanded and expanded state, the first and second edges 450, 452 are provided between/sandwiched between the stiff inner member 404 and the outer layer 402, and the slit 441 of the outer liner layer 408 is circumferentially disposed opposite (e.g., 180 degrees apart) from the slit 415 of the stiff inner member 404. But, in other examples, the slit 441 of the outer liner layer 408 is circumferentially disposed less than 180 degrees apart from the slit 415 of the stiff inner member 404. In the unexpanded state, the first edge 450 of the outer liner layer 406 is adjacent the second edge 452 of the outer liner layer 406. In some examples, the first edge 450 and the second edge 452 abut each other when the sheath is in the unexpanded state. In the example shown in FIG.7A, the first edge 450 and the second edge 452 of the outer liner layer 408 are separated by a minimal circumferential distance/gap when the sheath 400 is in the unexpanded state. In the example shown in FIG. 7B, as the sheath 400 expands spacing between the first edge 450 and the second edge 452 of the outer liner layer 408 increases to a circumferential distance as the outer liner layer 408 expands to accommodate the size of a medical device passing through the inner lumen 401. The maximum circumferential distance between the first edge 450 and the second edge 452 of the outer liner layer 408 is half the circumference of the stiff inner member 404. As such, in the example illustrated in FIGS. 7A-8, the distance in the expanded state is greater than a circumferential distance between the first edge 450 and second edge 452 when the sheath 400 is in the unexpanded state. The outer liner layer 408 extends about a smaller fraction of the circumference of the inner lumen 401 of the sheath 400 when in the expanded state than when in the unexpanded state. During expansion, the arc length between the first edge 450 and the second edge 452 of the outer liner layer 408 stays about constant while radius of a semicircle formed by the cross section of the stiff outer liner layer 406 increases. The outer liner layer 408 has a diameter of 0.219 inches when the sheath is in the unexpanded state. The outer liner layer 408 has a radial thickness of 0.0015 inches.

In the unexpanded and expanded state, at least a portion of the outer liner layer 408 disposed between the outer surface 424 of the stiff inner member 404 and the inner surface 414 of the outer layer 402. The outer liner layer 408 is coupled to the stiff inner member 404. The first edge 450 (or other suitable portion) of the outer liner layer 408 is coupled to the outer surface 424 of the stiff inner member 404 by reflow, although in other examples the first edge 450 (or other suitable portion) is coupled to the stiff inner member 404 by adhesive, co-extrusion, or any other bonding mechanism suitable to couple a lubricious layer to a stiffening shell. As illustrated in FIG. 7B, the inner liner layer 406 and the outer liner layer 408 together define the inner lumen 401 of the sheath 400 when the sheath 400 is in the expanded state. In the expanded state, the inner diameter of the central lumen of the sheath 400 (measured between the inner surface of the stiff inner member 404/inner liner layer 406 and the outer layer 402/outer liner layer 406 as illustrated in FIG. 7B) ranges from about 0.256 inches to about 0.384 inches. In some examples, the inner diameter of the expanded central lumen is about 0.320 inches. In some examples, the inner liner layer 406 and the outer liner layer 408 are each formed from a synthetic fluoropolymer such as Polytetrafluoroethylene (PTFE).

Although in the example shown in FIGS. 7A-8, the outer liner layer 408 has a diameter of about 0.219 inches, in other examples, the outer liner layer 408 has any diameter from about 0.175 to about 0.263 inches. In some examples, the outer liner layer 408 has any diameter up to a maximum outer diameter of the stiff inner member 404. Although the outer liner layer 408 has a thickness of about 0.0015 inches, in other examples, the outer liner layer 408 has any thickness from about 0.001 to about 0.005 inches or any other thickness suitable to resist folding upon movement between the unexpanded state and the expanded state. Although in the examples shown in FIGS. 7A-8, the outer liner layer 408 is spaced proximal of the distal end 412 of the outer layer 402 (e.g., 0.5 inches) and spaced distal of the proximal end 410 of the outer layer 402 (e.g., 0.5 inches), in some examples, the outer liner layer 408 is spaced any length up to 0.6 inches proximal of the distal end 412 of the outer layer 402 and up to 0.6 inches distal of the proximal end 410 of the outer layer 402. In some examples the outer liner layer 408 extends the entire length of the outer layer 402. Although the inner lumen 401 has a maximum diameter of about 0.320 inches when in the expanded state, in some examples, the inner lumen 401 has any maximum diameter ranging from about 0.256 inches to about 0.384 inches or any other diameter suitable to accept a prosthetic device. Although in the examples shown in FIGS. 7A-8, the inner liner layer 406 and the outer liner layer 408 are each is formed from a fluoropolymer such as Polytetrafluoroethylene (PTFE), in other examples the outer liner layer 406 and the outer liner layer 408 are each is formed from polyurethane such as Tecoflex or any other material suitable to provide a lubricious surface for a medical device passing through the sheath 400. Although in the example shown, the outer liner layer 408 includes a slit 441 and is coupled to a portion of the stiff inner member 404 (e.g., along the first edge 450), in some examples where the outer liner layer 406 is formed from an elastomer, the outer liner layer 406 is coupled to the stiff inner member 404 at least one location in addition to the first edge 450 (e.g., at second edge 452). In some examples the outer liner layer 408 is a continuous cylinder that is elastically radially expandable.

Like sheaths 10, 300 and 500, sheath 400 can be used in methods similar to those described above to deliver, remove, repair, and/or replace a prosthetic device. When using the sheath 400 depicted in FIGS. 7A-8, advancing an implant/medical device through the sheath 400 effects a radially directed outward force against the inner surface 434 of the inner liner layer 406, which moves the sheath 400 from the unexpanded state to the expanded state. Moving the sheath 400 from the unexpanded state to the expanded state pushes the inner liner layer 406 radially outward, increasing the gap between first edges 426, 438 and the second edges 428, 440 of the inner liner layer 406 and the stiff inner member 404 and exposing at least a portion of the outer liner layer 408 to the implant/medical device. At least one edge of the outer liner layer 408 moves circumferentially about the stiff inner member 404 such that a progressively smaller portion of the outer liner layer 408 overlaps with the stiff inner member 404 as the sheath 400 is moved from the unexpanded state to the expanded state. As such, the implant/medical device is advanced along the inner liner layer 406 when the sheath 400 is in the unexpanded condition, and advances along the inner liner layer 406 and the outer liner layer 408 locally expanded the sheath 400 from the unexpanded to the expanded state.

### EXEMPLARY ASPECTS

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: A sheath comprising: a radially expandable cylindrical outer layer having a proximal end and a distal end, and defining a cylindrically shaped lumen extending longitudinally between the proximal end and the distal end, and having an inner surface; and a plurality of sheath fins distributed circumferentially about the inner surface and coupled thereto, wherein each of the sheath fins extends along a length of the inner surface of the outer layer, wherein the sheath is movable between an unexpanded state and an expanded state, and where in the unexpanded state the sheath fins form a continuous surface of the lumen of the outer layer.

Example 2: The sheath according to any example herein, particularly example 1, wherein at least a portion of the sheath is configured to expand to the expanded state when a radial force is applied to the sheath fins from inside the lumen.

Example 3: The sheath according to any example herein, particularly examples 1 or 2, wherein at least a portion of the sheath is configured to retract to the unexpanded state when the radial force is not applied to the sheath fins.

Example 4: The sheath according to any example herein, particularly examples 1-3, wherein the sheath is configured to locally expand at a local axial location to the expanded state when a radial force is applied to the sheath fins from inside the lumen, wherein the sheath is configured to locally contract towards the unexpanded state when the radial force is no longer applied to the sheath fins from the inside of the lumen.

Example 5: The sheath according to any example herein, particularly examples 1-4, wherein the sheath fins have a greater stiffness than the outer layer.

Example 6: The sheath according to any example herein, particularly examples 1-5, wherein a longitudinal stiffness of the sheath is greater than the radial stiffness of the sheath.

Example 7: The sheath according to any example herein, particularly examples 1-6, wherein each of the sheath fins extend along a majority of a total length of the inner surface of the outer layer.

Example 8: The sheath according to any example herein, particularly examples 1-7, wherein each of the sheath fins extend along a total length of the inner surface of the outer layer.

Example 9: The sheath according to any example herein, particularly examples 1-8, wherein each of the sheath fins have an arcuate shape in cross-section.

Example 10: The sheath according to any example herein, particularly example 9, wherein each of the sheath fins have an arcuate-shaped outer surface and an arcuate-shaped inner surface, in cross-section.

Example 11: The sheath according to any example herein, particularly example 10, wherein a radius of the outer surface of each of the sheath fins and a radius of the corresponding inner surface of each of the sheath fins is the same.

Example 12: The sheath according to any example herein, particularly example 10, wherein a radius of the outer surface of each of the sheath fins is greater than a radius of the corresponding inner surface of each of the sheath fins.

Example 13: The sheath according to any example herein, particularly examples 1-12, wherein each of the sheath fins have an arcuate-shaped outer surface and a flat-shaped inner surface.

Example 14: The sheath according to any example herein, particularly examples 1-13, wherein a cross-sectional shape of each of the sheath fins is the same.

Example 15: The sheath according to any example herein, particularly examples 1-13, wherein a cross-sectional shape of at least one fin varies from a cross-sectional from at least one other fin.

Example 16: The sheath according to any example herein, particularly examples 1-15, wherein each of the sheath fins have a uniform radius about an outer circumference of the sheath.

Example 17: The sheath according to any example herein, particularly examples 1-16, wherein each of the sheath fins have a uniform radius about an inner circumference of the sheath.

Example 18: The sheath according to any example herein, particularly examples 1-17, wherein each of the sheath fins abut an adjacent one of each of the sheath fins when the sheath is in the unexpanded state.

Example 19: The sheath according to any example herein, particularly examples 1-18, wherein each of the sheath fins includes a longitudinally extending leading edge and a longitudinally extending trailing edge, wherein the leading edge of each of the sheath fins abuts a trailing edge of an adjacent one of the sheath fins when the sheath is in the unexpanded sate.

Example 20: The sheath according to any example herein, particularly example 19, wherein the leading and trailing edges define angled surfaces with respect to an outer and inner surface of each of the sheath fins.

Example 21: The sheath according to any example herein, particularly example 20, wherein the angled surfaces of the leading and trailing edges each extend in a plane that intersects with a longitudinal axis of the sheath.

Example 22: The sheath according to any example herein, particularly examples 19-21, wherein the leading edge of each of the sheath fins slides along the trailing edge of an adjacent fin when the sheath transitions between the unexpanded and the expanded state.

Example 23: The sheath according to any example herein, particularly examples 19-22, wherein at least one of the leading and the trailing edge of each of the sheath fins includes a surface feature for promoting sliding movement between adjacent sheath fins during expansion and contraction of the sheath.

Example 24: The sheath according to any example herein, particularly example 23, wherein the surface feature includes at least one of a groove, a channel, a surface treatment, and a lubricant.

Example 25: The sheath according to any example herein, particularly examples 1-24, wherein when the sheath expands from the unexpanded to the expanded state a circumferential spacing between adjacent sheath fins increases.

Example 26: The sheath according to any example herein, particularly example 25, wherein when the sheath expands from the unexpanded to the expanded state the circumferential spacing between adjacent sheath fins increases to form a gap between a each of the sheath fins.

Example 27: The sheath according to any example herein, particularly example 26, wherein a width of each of the sheath fins is measured between the leading and trailing edges of the fin, wherein in the expanded state a width of the gap formed between adjacent sheath fins is less than half the width of one of the adjacent sheath fins.

Example 28: The sheath according to any example herein, particularly example 26, wherein a width of each of the sheath fins is measured between the leading and trailing edges of the fin, wherein in the expanded state a width of the gap formed between adjacent sheath fins is half the width of one of the adjacent sheath fins.

Example 29: The sheath according to any example herein, particularly example 26, wherein a width of each of the sheath fins is measured between the leading and trailing edges of the fin, wherein in the expanded state a width of the gap formed between adjacent sheath fins is at least half the width of one of the adjacent sheath fins.

Example 30: The sheath according to any example herein, particularly examples 1-29, wherein in the unexpanded state each of the sheath fins is spaced apart from an adjacent sheath fins.

Example 31: The sheath according to any example herein, particularly example 30, wherein in the unexpanded state a width of the gap between adjacent sheath fins is less than half the of a width of one of the adjacent sheath fins.

Example 32: The sheath according to any example herein, particularly examples 1-31, wherein, each of the sheath fins have a uniform width, where the width of a fin is measured between a leading edge of a first fin and a trailing edge of an adjacent fin.

Example 33: The sheath according to any example herein, particularly examples 1-32, wherein the sheath fins have a plurality of widths.

Example 34: The sheath according to any example herein, particularly examples 1-33, wherein a combined thickness of the wall and the sheath fins is between about 0.04″ and about 0.07″.

Example 35: The sheath according to any example herein, particularly examples 1-34, wherein a combined thickness of the wall and the sheath fins is less than 0.06″ measured between an outer surface of the outer layer and an inner surface of at least one of the sheath fins.

Example 36: The sheath according to any example herein, particularly examples 1-35, wherein the plurality of sheath fins comprises at least four sheath fins.

Example 37: The sheath according to any example herein, particularly examples 1-36, wherein the plurality of sheath fins comprises at least eight sheath fins.

Example 38: The sheath according to any example herein, particularly examples 1-37, wherein an outer diameter of the sheath when in an unexpanded state ranges between about 0.20″ and about 0.30″.

Example 39: The sheath according to any example herein, particularly examples 1-38, wherein an outer diameter of the sheath when in an unexpanded state is about 0.24″.

Example 40: The sheath according to any example herein, particularly examples 1-39, wherein an outer diameter of the sheath when in an expanded state ranges between about 0.30″ and about 0.50″.

Example 41: The sheath according to any example herein, particularly examples 1-39, wherein an outer diameter of the sheath when in the expanded state is about 0.40″.

Example 42: The sheath according to any example herein, particularly examples 1-41, wherein the sheath has a uniform radius about the circumference of the sheath when in the expanded state.

Example 43: The sheath according to any example herein, particularly examples 1-42, wherein the sheath has a uniform radius about the circumference of the sheath when in the unexpanded state.

Example 44: The sheath according to any example herein, particularly examples 1-43, wherein the sheath symmetrically expands in the radial direction during expansion of the sheath between the unexpanded and the expanded state, wherein a portion of the outer layer extending between adjacent fins stretches and/or expands during expansion of the outer layer, wherein a portion of the outer layer coupled to the sheath fins does not stretch and/or expand during expansion of the outer layer.

Example 45: The sheath according to any example herein, particularly examples 1-44, wherein the sheath fins are formed from plastic.

Example 46: The sheath according to any example herein, particularly examples 1-45, wherein the sheath fins are formed from silicone.

Example 47: The sheath according to any example herein, particularly examples 1-46, wherein the outer layer is formed from an elastomer material.

Example 48: The sheath according to any example herein, particularly examples 1-47, wherein the sheath fins are integrally formed with the outer layer.

Example 49: The sheath according to any example herein, particularly examples 1-48, wherein the sheath fins are coextruded with the outer layer.

Example 50: The sheath according to any example herein, particularly examples 1-49, wherein the each of the sheath fins are fixedly coupled to the outer layer.

Example 51: The sheath according to any example herein, particularly example 50, wherein each of the sheath fins are coupled to the outer layer by adhesive

Example 52: The sheath according to any example herein, particularly examples 50 or 51, wherein each of the sheath fins are bonded to the outer layer by a molding process.

Example 53: The sheath according to any example herein, particularly examples 1-52, further comprising an end cap coupled to the distal end of the sheath.

Example 54: The sheath according to any example herein, particularly example 53, wherein the end cap is formed from plastic.

Example 55: The sheath according to any example herein, particularly examples 53 and 54, wherein the end cap is integrally formed with the outer layer.

Example 56: The system according to any example herein, particularly examples 1-55, wherein the sheath is an introducer sheath used for delivery of an implantable medical device.

Example 57: A sheath system comprising: a sheath comprising: a radially expandable cylindrical outer layer having a proximal end and a distal end, and defining a cylindrically shaped lumen extending longitudinally between the proximal end and the distal end, and having an inner surface; and a plurality of sheath fins distributed circumferentially about the inner surface and coupled thereto, wherein each of the sheath fins extends along a length of the inner surface of the outer layer, wherein the sheath is movable between an unexpanded state and an expanded state, and wherein the unexpanded state the sheath fins form a continuous surface of the lumen of the outer layer; and an introducer sheath hub having a central lumen and a distal end, wherein the distal end of the introducer sheath hub is coupled to the proximal end of the introducer sheath, and where the central lumen of the sheath hub is coaxial with the central lumen of the introducer sheath.

Example 58: The system of claim 57, further comprising a strain relief tube having an central lumen a proximal end and a distal end, wherein a portion of sheath is disposed within the central lumen of the strain relief tube, wherein the proximal end of the strain relief tube is coupled to the introducer sheath hub and the distal end of the strain relief tube is coupled to the sheath.

Example 59: A sheath comprising: a radially expandable tube body having a proximal end a distal end, and defining a cylindrically shaped lumen extending longitudinally between the proximal end and the distal end, and having a wall thickness that extends between the proximal end and the distal end, and a plurality of wires distributed circumferentially within the side wall of the tube body, wherein each of the wires extend longitudinally between the proximal end and the distal end of the tube body, wherein the sheath is movable between an unexpanded state and an expanded state, and wherein the lumen has a uniform cylindrical shape in the unexpanded state, and the expanded state.

Example 60: The sheath according to any example herein, particularly examples 59, wherein at least a portion of the sheath is configured to expand to the expanded state when a radial force is applied to the tube body from inside the lumen.

Example 61: The sheath according to any example herein, particularly examples 59 or 60, wherein at least a portion of the sheath is configured to retract to the unexpanded state when the radial force is not applied to the tube body.

Example 62: The sheath according to any example herein, particularly examples 59-61, wherein the sheath is configured to locally expand at a local axial location to the expanded state when a radial force is applied to the tube body from inside the lumen, wherein the sheath is configured to locally contract towards the unexpanded state when the radial force is no longer applied to the tube body from the inside of the lumen.

Example 63: The sheath according to any example herein, particularly examples 59-62, wherein the wires have a greater stiffness than the expandable tube.

Example 64: The sheath according to any example herein, particularly examples 59-63, wherein each of the wires extend along a majority of a total length of the inner surface of the outer layer.

Example 65: The sheath according to any example herein, particularly examples 59-64, wherein each of the wires extend along a total length of the inner surface of the outer layer.

Example 66: The sheath according to any example herein, particularly examples 59-65, wherein a longitudinal stiffness of the sheath is greater than the radial stiffness of the sheath.

Example 67: The sheath according to any example herein, particularly examples 59-66, wherein the wires are evenly distributed about the circumference of the sheath.

Example 68: The sheath according to any example herein, particularly examples 59-67, wherein the sheath is configured to expand in a circumferentially and radially uniform manner.

Example 69: The sheath according to any example herein, particularly examples 59-68, wherein the sheath is formed from silicone.

Example 70: The sheath according to any example herein, particularly examples 59-69, wherein the wires are each formed from stainless steel.

Example 71: The sheath according to any example herein, particularly examples 59-70, wherein each wire has a uniform circumference.

Example 72: The sheath according to any example herein, particularly examples 59-71, wherein each wire has a diameter is about 0.020″.

Example 73: The sheath according to any example herein, particularly examples 59-71, wherein each wire has a diameter of about 0.025″.

Example 74: The sheath according to any example herein, particularly examples 59-73, wherein each wire is fully embedded in the side wall of the tube body

Example 75: The sheath according to any example herein, particularly examples 59-73, wherein each wire is partially embedded in the side wall of the tube body and partially exposed to the inner lumen.

Example 76: The sheath according to any example herein, particularly examples 59-75, wherein the plurality of wires comprises eight wires.

Example 77: The sheath according to any example herein, particularly examples 59-76, wherein the diameter when in an unexpanded state is 0.24″.

Example 78: The sheath according to any example herein, particularly examples 59-77, wherein the diameter when in the expanded state is 0.40″.

Example 79: The sheath according to any example herein, particularly examples 59-78, wherein the sheath has a uniform radius about the circumference of the sheath when in the expanded state

Example 80: The sheath according to any example herein, particularly examples 59-79, wherein the sheath has a uniform radius about the circumference of the sheath when in the unexpanded state.

Example 81: The sheath according to any example herein, particularly examples 59-80, further comprising an end cap coupled to the distal end of the sheath.

Example 82: The sheath according to any example herein, particularly example 81, wherein the end cap is formed from plastic.

Example 83: The sheath according to any example herein, particularly examples 81 and 82, wherein the end cap is integrally formed with the outer layer.

Example 84: The sheath according to any example herein, particularly examples 59-83, wherein the wires are integrally formed with the tube body.

Example 85: The sheath according to any example herein, particularly examples 59-84, wherein the wires are coextruded with the tube body.

Example 86: The sheath according to any example herein, particularly examples 59-83, wherein the each of the wires are fixedly coupled to the tube body.

Example 87: The sheath according to any example herein, particularly examples 85, wherein the wires are coupled to the tube by adhesive.

Example 88: The sheath according to any example herein, particularly examples 59-87, wherein the wires are coupled to the tube by a molding process.

Example 89: The sheath according to any example herein, particularly examples 59-88, wherein the sheath is an introducer sheath used for delivery of an implantable medical device.

Example 90: A sheath system comprising: a sheath comprising: a radially expandable tube body having a proximal end a distal end, and defining a cylindrically shaped lumen extending longitudinally between the proximal end and the distal end, and having a wall thickness that extends between the proximal end and the distal end, and a plurality of wires distributed circumferentially within the sidewall of the tube body, wherein each of the wires extend longitudinally between the proximal end and the distal end of the tube body, wherein the sheath is movable between an unexpanded state and an expanded state, and wherein the inner lumen is a uniform cylindrical shape in the unexpanded and the expanded state; and an introducer sheath hub having a central lumen and a distal end, wherein the distal end of the introducer sheath hub is coupled to the proximal end of the introducer sheath, and wherein the central lumen of the sheath hub is coaxial with the central lumen of the introducer sheath.

Example 91: The system according to any example herein, particularly examples 90, further comprising a strain relief tube having a central lumen, a proximal end, and a distal end, wherein a portion of sheath is disposed within the central lumen of the strain relief tube, and wherein the proximal end of the strain relief tube is coupled to the introducer sheath hub and the distal end of the strain relief tube is coupled to the sheath.

Example 92: A method of delivering a medical device (and/or a method of expanding an introducer sheath by a passing medical device) comprising: when delivering the medical device to a patient, inserting an introducer sheath into a blood vessel, the introducer sheath comprising: a radially expandable cylindrical outer layer having a proximal end and a distal end, and defining a cylindrically shaped lumen extending longitudinally between the proximal end and the distal end, and having an inner surface; and a plurality of sheath fins distributed circumferentially about the inner surface and coupled thereto, wherein each of the sheath fins extend along a length of the inner surface of the outer layer of the introducer sheath, where the sheath is movable between an unexpanded state and an and an expanded state, in the unexpanded state the sheath fins form an inner surface of the lumen of the outer layer; advancing a medical device through the lumen along an axis of the sheath and toward the distal end of the lumen; and expanding the lumen of the sheath while advancing the medical device through the introducer sheath, wherein the sheath expands symmetrically in the radial direction.

Example 93: The method according to any example herein, particularly example 92, wherein advancing the medical device through the sheath moves at least a portion of the sheath from the unexpanded state to the expanded state.

Example 94: The method according to any example herein, particularly examples 92 and 93, wherein advancing a medical device through the lumen further comprises advancing the medical device from the proximal end of the sheath of the distal end of the sheath.

Example 95: The method according to any example herein, particularly examples 92-94, wherein advancing the medical device through the sheath further comprises advancing the medical device from the distal end of the sheath to the proximal end of the sheath.

Example 96: The method according to any example herein, particularly examples 92-95, wherein an outer diameter of the sheath when in an unexpanded state ranges between about 0.20″ and about 0.30″.

Example 97: The method according to any example herein, particularly examples 92-96, wherein an outer diameter of the sheath when in an unexpanded state is about 0.24″.

Example 98: The method according to any example herein, particularly examples 92-97, wherein an outer diameter of the sheath when in an expanded state ranges between about 0.30″ and about 0.50″.

Example 99: The method according to any example herein, particularly examples 92-98, wherein the expanded state is wherein an outer diameter of the sheath when in an expanded state ranges between about 0.30″ and about 0.50″.

Example 100: The method according to any example herein, particularly examples 92-99, wherein an outer diameter of the sheath when in the expanded state is about 0.40″.

Example 101: The method according to any example herein, particularly examples 92-100, further comprising removing the introducer sheath from the blood vessel.

Example 102: The method according to any example herein, particularly examples claim 92-101, wherein advancing the medical device through the lumen further comprises radially displacing the fins with the medical device.

Example 103: The method according to any example herein, particularly examples claim 92-102, wherein advancing the medical device through the lumen causes circumferential separation between each of the plurality of sheath fins.

Example 104: A method of delivering a medical device (and/or a method expanding an introducer sheath by a passing medical device) comprising: when delivering the medical device to a patient, inserting an introducer sheath into a blood vessel, the introducer sheath comprising: a radially expandable tube body having a proximal end a distal end, and defining a cylindrically shaped lumen extending longitudinally between the proximal end and the distal end, and having a wall thickness that extends between the proximal end and the distal end, and a plurality of wires distributed circumferentially within the thickness of the tube body, wherein each of the wires extend longitudinally between the proximal end and the distal end of the tube body, wherein the sheath is movable between an unexpanded state and an expanded state, and wherein the inner lumen is a uniform cylinder in the unexpanded state, and the expanded state; advancing a medical device through the lumen along an axis of the sheath and toward the distal end of the lumen; and expanding the lumen of the sheath while advancing the medical device through the introducer sheath, wherein the sheath expands symmetrically in the radial direction.

Example 105: The method according to any example herein, particularly example 104, wherein advancing the medical device through the sheath moves at least a portion of the sheath from the unexpanded state to the expanded state.

Example 106: The method according to any example herein, particularly examples 104 and 105, wherein advancing a medical device through the lumen further comprises advancing the medical device from the proximal end of the sheath of the distal end of the sheath.

Example 107: The method according to any example herein, particularly examples 104-106, wherein advancing the medical device through the sheath further comprises advancing the medical device from the distal end of the sheath to the proximal end of the sheath.

Example 108: The method according to any example herein, particularly examples 104-107, wherein an outer diameter of the sheath when in an unexpanded state ranges between about 0.20″ and about 0.30″.

Example 109: The method according to any example herein, particularly examples 104-108, wherein an outer diameter of the sheath when in an unexpanded state is about 0.24″.

Example 110: The method according to any example herein, particularly examples 104-109, wherein an outer diameter of the sheath when in an expanded state ranges between about 0.30″ and about 0.50″.

Example 111: The method according to any example herein, particularly examples 104-110, wherein an outer diameter of the sheath when in the expanded state is about 0.40″.

Example 112: The method according to any example herein, particularly examples 104-111, further comprising removing the introducer sheath from the blood vessel.

Example 113: The method according to any example herein, particularly examples 104-112, wherein advancing the medical device through the lumen further comprises radially displacing the wires with the medical device.

Example 114: The method according to any example herein, particularly examples 104-113, wherein advancing the medical device through the lumen causes circumferential separation between each of the plurality of wires.

Example: 115: A sheath comprising: a radially expandable cylindrical outer layer having a proximal end and a distal end, and defining a cylindrically shaped lumen extending longitudinally between the proximal end and the distal end, and having an inner surface; a stiff inner member having a proximal end and a distal end, an inner surface, and an outer surface opposite and spaced apart from the inner surface of the stiff inner member and each extending between the proximal end and the distal end of the stiff inner member, and the stiff inner member provided within at least a portion of the lumen of the outer layer; an inner liner layer coupled to the inner surface of the stiff inner member; and an outer liner layer disposed between the outer surface of the stiff inner member and the inner surface of the outer layer, wherein the sheath is movable between an unexpanded state and an expanded state, wherein in the unexpanded state, the inner liner layer forms a surface defining an inner lumen of the sheath, wherein in the expanded state, the inner liner layer and the outer liner layer each define a portion of the inner lumen of the sheath, and wherein the outer liner layer at least partially radially overlaps the stiff inner member in the unexpanded and expanded states.

Example 116: The sheath according to any example herein, particularly example 115, wherein the coefficient of friction of the inner liner layer, and the outer liner layer are each less than the coefficient of friction of the stiff inner member.

Example 117: The sheath according to any example herein, particularly examples 115 or 116, wherein the inner liner layer and the outer liner layer form a lubricious surface about the entire circumference of the inner lumen when the sheath is in the expanded state.

Example 118: The sheath according to any example herein, particularly examples 115-117, wherein the stiff inner member, the inner liner layer, and the outer liner layer each extend between a longitudinal position spaced from the proximal and distal ends of the outer layer (e.g., the inner and outer liner layer are spaced 0.5 inches proximal of the distal end of the outer layer and 0.5 inches distal of the proximal end of the outer layer).

Example 119: The sheath according to any example herein, particularly examples 115-118, wherein the stiff inner member includes a slit that extends between the proximal end and the distal end and defines a first edge and a second edge.

Example 120: The sheath according to any example herein, particularly example 119, wherein the first edge of the stiff inner member is adjacent the second edge of the stiff inner member when the sheath is in the unexpanded state.

Example 121: The sheath according to any example herein, particularly examples 119 or 120, wherein the first edge and the second edge of the stiff inner member are separated by a circumferential distance when the sheath is in the expanded state, where the circumferential distance in the expanded state is greater than a circumferential distance between the first and second edge when in the unexpanded state.

Example 122: The sheath according to any example herein, particularly examples 115-121, wherein the stiff inner member extends about a smaller fraction of the circumference of the inner lumen when in the expanded state than when in the unexpanded state.

Example 123: The sheath according to any example herein, particularly examples 115-122, wherein the inner liner layer includes a slit that extends between the proximal end and the distal end and defines a first edge and a second edge.

Example 124: The method according to any example herein, particularly example 123, wherein the first edge of the inner liner layer is adjacent the second edge of the inner liner layer when the sheath is in the unexpanded state.

Example 125: The sheath according to any example herein, particularly examples 123-124, wherein the first edge and the second edge of the inner liner layer are separated by a circumferential distance when the sheath is in the expanded state, where the circumferential distance in the expanded state is greater than a circumferential distance between the first and second edge when in the unexpanded state.

Example 126: The sheath according to any example herein, particularly examples 115-125, wherein the inner liner layer extends about a smaller fraction of the circumference of the sheath when the sheath is in the expanded state than when the sheath is in the unexpanded state.

Example 127: The sheath according to any example herein, particularly examples 115-126, wherein the outer liner layer includes a slit that extends between the proximal end and the distal end defining a first edge and a second edge.

Example 128: The method according to any example herein, particularly example 127, wherein the first edge of the outer liner layer is coupled to the outer surface of the stiff inner member.

Example 129: The method according to any example herein, particularly examples 127 or 128, wherein the first edge of the outer liner layer is adjacent the second edge of the outer liner layer when the sheath is in the unexpanded state.

Example 130: The sheath according to any example herein, particularly examples 127-129, wherein the first edge and the second edge of the outer liner layer are separated by a circumferential distance when the sheath is in the expanded state, where the circumferential distance in the expanded state is greater than a circumferential distance between the first and second edge when in the unexpanded state.

Example 131: The sheath according to any example herein, particularly examples 115-130, wherein the outer liner layer extends about a smaller fraction of the circumference of the sheath when the sheath is in the expanded state than when the sheath is in the unexpanded state.

Example 132: The sheath according to any example herein, particularly examples 115-131, wherein the outer liner layer is an elastomer.

Example 133: The sheath according to any example herein, particularly example 132, wherein the outer liner layer is a continuous cylinder.

Example 134: A sheath system comprising: a sheath comprising: a radially expandable cylindrical outer layer having a proximal end and a distal end, and defining a cylindrically shaped lumen extending longitudinally between the proximal end and the distal end, and having an inner surface; a stiff inner member having a proximal end and a distal end, an inner surface, and an outer surface opposite and spaced apart from the inner surface of the stiff inner member and each extending between the proximal end and the distal end of the stiff inner member, and the stiff inner member provided within at least a portion of the lumen of the outer layer; an inner liner layer coupled to the inner surface of the stiff inner member, and an outer liner layer disposed between the outer surface of the stiff inner member and the inner surface of the outer layer; and an introducer sheath hub having a central lumen and a distal end, wherein the distal end of the introducer sheath hub is coupled to the proximal end of the sheath, and where the central lumen of the sheath hub is coaxial with the central lumen of the sheath, wherein the sheath is movable between an unexpanded state and an expanded state, wherein in the unexpanded state, the inner liner layer forms a surface defining an inner lumen of the sheath, wherein in the expanded state, the inner liner layer and the outer liner layer each define a portion of the inner lumen of the sheath, and wherein the outer liner layer at least partially radially overlaps the stiff inner member in the unexpanded and expanded states.

Example 135: The system according to any example herein, particularly examples 134, wherein the coefficient of friction of the inner liner layer, and the outer liner layer are each less than the coefficient of friction of the stiff inner member.

Example 136: The system according to any example herein, particularly examples 134 or 135, wherein the inner liner layer and the outer liner layer form a lubricious surface about the circumference of the inner lumen when the sheath is in the expanded state.

Example 137: The system according to any example herein, particularly examples 134-136, wherein the stiff inner member, the inner liner layer, and the outer liner layer each extend between a longitudinal position spaced from the proximal and distal ends of the outer layer (e.g., the inner and outer liner layer are spaced 0.5 inches proximal of the distal end of the outer layer and 0.5 inches distal of the proximal end of the outer layer).

Example 138: A method of delivering a medical device (and/or a method of expanding an introducer sheath by a passing medical device) comprising: when delivering the medical device to a patient, inserting an introducer sheath into a blood vessel, the introducer sheath comprising: a radially expandable cylindrical outer layer having a proximal end and a distal end, and defining a cylindrically shaped lumen extending longitudinally between the proximal end and the distal end, and having an inner surface; a stiff inner member having a proximal end and a distal end, an inner surface, and an outer surface opposite and spaced apart from the inner surface of the stiff inner member and each extending between the proximal end and the distal end of the stiff inner member, and the stiff inner member provided within at least a portion of the lumen of the outer layer; an inner liner layer coupled to the inner surface of the stiff inner member, and an outer liner layer disposed between the outer surface of the stiff inner member and the inner surface of the outer layer, wherein the sheath is movable between an unexpanded state and an expanded state, wherein in the unexpanded state, the inner liner layer forms a surface defining an inner lumen of the sheath, wherein in the expanded state, the inner liner layer and the outer liner layer each define a portion of the inner lumen of the sheath, and wherein the outer liner layer at least partially radially overlaps the stiff inner member in the unexpanded and expanded states; advancing a medical device through the lumen along an axis of the sheath and toward the distal end of the lumen; and expanding the lumen of the sheath while advancing the medical device through the introducer sheath, wherein the sheath expands in the radial direction.

Example 139: The method according to any example herein, particularly examples 138, wherein expanding the lumen comprises moving the sheath from the unexpanded state to the expanded state.

Example 140: The method according to any example herein, particularly examples 138 or 139, wherein advancing the medical device through the sheath moves at least a portion of the sheath from the unexpanded state to the expanded state.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

What is claimed is:

1. A sheath comprising:
a radially expandable cylindrical outer layer having a proximal end and a distal end, and defining a cylindrically shaped lumen extending longitudinally between the proximal end and the distal end, and having an inner surface; and
a plurality of sheath fins distributed circumferentially about the inner surface and coupled thereto,
wherein each of the sheath fins extends along a length of the inner surface of the outer layer,
wherein the sheath is movable between an unexpanded state and an expanded state, and where in the unexpanded state the sheath fins form a continuous surface of the lumen of the outer layer.

2. The sheath of claim 1, wherein the sheath fins have a greater stiffness than the outer layer.

3. The sheath of claim 1, wherein a longitudinal stiffness of the sheath is greater than the radial stiffness of the sheath.

4. The sheath of claim 1, wherein each of the sheath fins extend along at least a majority of a total length of the inner surface of the outer layer.

5. The sheath of claim 1, wherein each of the sheath fins have an arcuate-shaped outer surface and an arcuate-shaped inner surface, in cross-section.

6. The sheath of claim 1, wherein each of the sheath fins have an arcuate-shaped outer surface and a flat-shaped inner surface.

7. The sheath of claim 1, wherein each of the sheath fins includes a longitudinally extending leading edge and a longitudinally extending trailing edge,
wherein the leading edge of each of the sheath fins abuts a trailing edge of an adjacent one of the sheath fins when the sheath is in the unexpanded sate.

8. The sheath of claim 7, wherein at least one of the leading and the trailing edge of each of the sheath fins includes a surface feature for promoting sliding movement between adjacent sheath fins during expansion and contraction of the sheath.

9. The sheath of claim 1, wherein when the sheath expands from the unexpanded to the expanded state the circumferential spacing between adjacent sheath fins increases to form a gap between each of the sheath fins.

10. A method of delivering a medical device and expanding an introducer sheath comprising:
providing an introducer sheath comprising:
a radially expandable cylindrical outer layer having a proximal end and a distal end, and defining a cylindrically shaped lumen extending longitudinally between the proximal end and the distal end, and having an inner surface; and
a plurality of sheath fins distributed circumferentially about the inner surface and coupled thereto,
wherein each of the sheath fins extends along a length of the inner surface of the outer layer of the introducer sheath, where the sheath is movable between an unexpanded state and an and an expanded state, in the unexpanded state the sheath fins form an inner surface of the lumen of the outer layer;
advancing a medical device through the lumen along an axis of the sheath and toward the distal end of the lumen; and
expanding the lumen of the sheath while advancing the medical device through the introducer sheath causing circumferential separation between each of the plurality of sheath fins,
wherein the sheath expands symmetrically in the radial direction.

11. A sheath comprising:
a radially expandable cylindrical outer layer having a proximal end and a distal end, and defining a cylindrically shaped lumen extending longitudinally between the proximal end and the distal end, and having an inner surface;
a stiff inner member having a proximal end and a distal end, an inner surface, and an outer surface opposite and spaced apart from the inner surface of the stiff inner member and each extending between the proximal end and the distal end of the stiff inner member, and the stiff inner member provided within at least a portion of the lumen of the outer layer;
an inner liner layer coupled to the inner surface of the stiff inner member; and
an outer liner layer disposed between the outer surface of the stiff inner member and the inner surface of the outer layer,
wherein the sheath is movable between an unexpanded state and an expanded state,
wherein in the unexpanded state, the inner liner layer forms a surface defining an inner lumen of the sheath,
wherein in the expanded state, the inner liner layer and the outer liner layer each define a portion of the inner lumen of the sheath, and
wherein the outer liner layer at least partially radially overlaps the stiff inner member in the unexpanded and expanded states.

12. The sheath of claim 11, wherein the coefficient of friction of the inner liner layer, and the outer liner layer are each less than the coefficient of friction of the stiff inner member.

13. The sheath of claim 11, wherein the inner liner layer and the outer liner layer form a lubricious surface about the circumference of the inner lumen when the sheath is in the expanded state.

14. The sheath of claim 11, wherein the stiff inner member includes a slit that extends between the proximal end and the distal end and defines a first edge and a second edge, wherein the first edge of the stiff inner member is adjacent the second edge of the stiff inner member when the sheath is in the unexpanded state, wherein the first edge and the second edge of the stiff inner member are separated by a circumferential distance when the sheath is in the expanded state, where the circumferential distance in the expanded state is greater than a circumferential distance between the first and second edge when in the unexpanded state.

15. The sheath of claim 11, wherein the stiff inner member extends about a smaller fraction of the circumference of the inner lumen when in the expanded state than when in the unexpanded state.

16. The sheath of claim 11, wherein the inner liner layer includes a slit that extends between the proximal end and the distal end and defines a first edge and a second edge, wherein the first edge and the second edge of the inner liner layer are separated by a circumferential distance when the sheath is in the expanded state, where the circumferential distance in the expanded state is greater than a circumferential distance between the first and second edge when in the unexpanded state.

17. The sheath of claim 11, wherein the outer liner layer includes a slit that extends between the proximal end and the distal end defining a first edge and a second edge.

18. The sheath of claim 17, wherein the first edge of the outer liner layer is coupled to the outer surface of the stiff inner member.

19. The sheath of claim 17, wherein the first edge of the outer liner layer is adjacent the second edge of the outer liner layer when the sheath is in the unexpanded state, wherein the first edge and the second edge of the outer liner layer are separated by a circumferential distance when the sheath is in the expanded state, where the circumferential distance in the expanded state is greater than a circumferential distance between the first and second edge when in the unexpanded state.

20. The sheath of claim 11, wherein the outer liner layer extends about a smaller fraction of the circumference of the sheath when the sheath is in the expanded state than when the sheath is in the unexpanded state.
